(19) United States
(12) Patent Application Publication
(10) Pub. No.: US 2019/0049473 A1
(43) Pub. Date: Feb. 14, 2019

(54) METHODS AND COMPOSITIONS FOR USING SOLVATOCHROMIC DYES TO DETECT THE PRESENCE OF HOMEOPATHIC POTENCIES

(71) Applicant: STANDARD HOMEOPATHIC COMPANY, Los Angeles, CA (US)

(72) Inventor: CARTWRIGHT Steven J., Los Angeles, CA (US)

(73) Assignee: STANDARD HOMEOPATHIC COMPANY, Los Angeles, CA (US)

(21) Appl. No.: 16/080,365

(22) PCT Filed: Feb. 28, 2017
(86) PCT No.: PCT/US17/20013

§ 371 Date: Aug. 28, 2018

Related U.S. Application Data
(60) Provisional application No. 62/300,864, filed on Feb. 28, 2016.

(51) Int. Cl. G01N 33/94; G01N 21/64; G01N 21/31; G01N 21/03

(52) U.S. Cl. CPC G01N 33/94; G01N 21/6428; C09B 23/102; G01N 21/03; G01N 21/31

(57) ABSTRACT

A method is provided for obtaining a characteristic of a homeopathic preparation, particularly its potency. In particular aspects, solvatochromic dyes may be used for detection of a homeopathic preparation.

This application claims the benefit of U.S. Provisional Application No. 62/300,864, filed Feb. 28, 2016.

### BACKGROUND

### 1. Field of the Invention

Disclosed herein are methods and compositions for obtaining characteristics of a homeopathic preparation. More specifically, reproducible methods and compositions for the quantitative characterization of homeopathic preparations employing solvatochromic dyes are disclosed.

### 2. Description of Related Art

Homeopathy is healing the sick by using substances capable of causing the same symptoms, syndromes, and conditions when administered to healthy people (Homeopathic Pharmacopoeia of the United States). Any substance may be considered a homeopathic preparation if it is known to mimic the symptoms, syndromes, or conditions in healthy people to whom it is administered.

A complication in the field of homeopathy is the lack of reproducible quality control for in-process or finished homeopathic products. Homeopathic industries are coming under increasing pressure to upgrade their quality control practices. However, this has proven difficult in light of the nature of homeopathic preparations, and inability of standard chemical based test methods to characterize in-process or finished products.

Various types of tests have been instituted in attempts to assure the consistent quality of homeopathic preparations, including a variety of chemical analyses. However, known optical and electrical methods to ensure the quality of homeopathic preparations have proven ineffective. Therefore, there remains a need for a reliable method for obtaining characteristics of homeopathic preparations.

### SUMMARY OF THE INVENTION

The inventors surprisingly determined that polar and polarizable molecules may be used as supramolecular chemical probes of serially diluted and agitated solutions. In some embodiments, a polar and polarizable molecule comprises a conjugated it-electron system through which negative charge may be delocalized. In further embodiments, the structure of a polar and polarizable molecule changes in response to solvent pH. In some embodiments, a polar molecule is a molecule with a molecule with a non-zero dipole moment. In some embodiments, a polar molecule exhibits non-uniform positive and negative charge distribution. In further embodiments, a polar molecule is a hydrocarbon bearing at least one non-carbon, non-hydrogen atom. In some aspects, a polarizable molecule is a molecule through which electron density or charge may be delocalized. In some embodiments, a polarizable molecule is a molecule for which more than one resonance structure can be drawn.

In some embodiments a polar and polarizable molecule is a dye or pigment. In some aspects, a polar and polarizable molecule is a solvatochromic dye. In further embodiments, a polar and polarizable molecule is a halochromic dye. A solvatochromic dye is a molecule whose color depends on the polarity of a solvent in which it is dissolved or mixed. A halochromic dye is a molecule whose color depends on the pH of the solvent in which it is dissolved or mixed.

Potencies influence the supramolecular chemistry of polar and polarizable molecules and enhance molecular aggregation or disaggregation. Electronic spectroscopy can be used to examine the interaction and influence of potencies with the supramolecular chemistry of polar and polarizable molecules by following changes in dye absorbance across the visible spectrum. Spectral variations may then be correlated to the effect of a homeopathic potency. The methods disclosed herein may be used to examine the effect of a number of potencies of homeopathic medicines, including but not limited to glycerol. Potencies include 6c, 12c, 15c, 200c, 1M, 10M, 50M and CM or a range of homeopathic medicines disclosed therein.

In some aspects, a method of quantifying a potency effect of a homeopathic potency comprises the steps of obtaining a sample solution by adding a homeopathic potency solution to a dye solution comprising a polar and polarizable molecule in a solvent; obtaining a reference solution comprising an equivalent amount of the dye solution; obtaining a spectrum of the sample solution; obtaining a spectrum of the reference solution; and obtaining a difference spectrum. In some embodiments, the maximum value of the difference spectrum correlates with the dipole moment of the polar and polarizable molecule. In some embodiments, the difference spectrum is the difference between the sample solution spectrum and the reference solution spectrum. In some embodiments, the reference solution spectrum is a control. The method of quantifying a potency effect may further comprise the step of identifying a maximum value of the difference spectrum. The homeopathic potency solution, polar and polarizable molecule solution, reference solution, and/or sample solution may be incubated for a period of up to 20 days prior to use or spectral determination thereof. In some embodiments, the polar and polarizable molecule is a solvatochromic dye. In further embodiments, the polar and polarizable molecule is a halochromic dye. In some embodiments there is a method of quantifying a homeopathic potency effect comprising 1, 2, 3, 4, 5, or 6 of the following steps: obtaining a sample solution by adding a homeopathic potency solution to a dye solution comprising a polar and polarizable molecule in a solvent; obtaining a reference solution comprising an equivalent amount of the dye solution of a previous step; obtaining a spectrum of the sample solution; obtaining a spectrum of the reference solution; obtaining a difference spectrum, wherein the difference spectrum is the difference between the sample solution spectrum and the reference solution spectrum; and/or, quantifying the potency effect of the homeopathic potency solution by identifying a maximum value of the difference spectrum. In other embodiments, there is a method comprising measuring a difference spectrum between a sample and a control and/or quantifying the potency effect of the homeopathic potency solution by identifying a maximum value of the difference spectrum.

In some embodiments, sample and reference solution spectra are obtained at a number of time intervals. These spectra are then used to determine difference spectra for each time interval. The maximum value of each difference spectra may then be plotted against time in order to monitor the change in difference spectra maxima. The plot of each difference spectra maximum versus time may be used to monitor the rate of change of difference spectra maxima.

A homeopathic potency solution may prepared by a serial dilution of more than 100²⁰⁰. In some aspects, a homeopathic potency solution is prepared by a serial dilution of up to Ser. No. 10/050,000. In some embodiments, a homeopathic potency solution may be prepared by a serial dilution of between 100⁶ to 100¹⁰⁰⁰⁰⁰. In certain embodiments, a homeopathic potency solution may be prepared by a serial dilution of about 100⁶, 100¹⁰, 100²⁰, 100³⁰, 100⁴⁰, 100⁵⁰, 100⁶⁰, 100⁷⁰, 100⁸⁰, 100⁹⁰, 100¹⁰⁰, 100²⁰⁰, 100³⁰⁰, 100⁴⁰⁰, 100⁵⁰⁰, 100⁶⁰⁰, 100⁷⁰⁰, 100⁸⁰⁰, 100⁹⁰⁰, 100¹⁰⁰⁰, 100⁵⁰⁰⁰, 100¹⁰⁰⁰⁰, 100²⁰⁰⁰⁰, 100³⁰⁰⁰⁰ 100⁴⁰⁰⁰⁰, 100⁵⁰⁰⁰⁰, 100⁶⁰⁰⁰⁰, 100⁷⁰⁰⁰⁰, 100⁸⁰⁰⁰⁰, 100⁹⁰⁰⁰⁰, 100¹⁰⁰⁰⁰⁰, or any range derivable therein. In some aspects, the polar and polarizable molecule is a negatively solvatochromic dye. In other aspects, the polar and polarizable molecule is a positively solvatochromic dye. In further aspects, the polar and polarizable molecule is a halochromic dye.

In some aspects, a negatively solvatochromic dye is ET33, ET30, or BM. In some aspects, a positively solvatochromic dye is BDN, BDF, NR, or PB. In some aspects, a halochromic dye is 6-amino-2-naphthoic acid (6ANA), 6-amino-2-anthracenic acid (6AAA), phenolphthalein, thymol blue, phenol red, bromothymol blue, methyl red, bromocresol green, methyl orange, or thymol blue. In certain embodiments, one or more of the dyes disclosed in this paragraph is specifically not used. Solvents that may be used for determining the potency effect include both ionic solvents and nonionic solvents. Non-limiting examples of nonionic solvents include water, ethanol, tert-butyl alcohol, dimethylsulfoxide. Non-limiting examples of ionic solvents include 1-ethyl-3-methylimidazolium acetate, 1-butyl-3-methylimidazolium hexafluorophosphate, benzyldimethyltetradecylammonium chloride, tetrabutylphosphonium methanesulfonate, 1-butyl-4-methylpyridinium hexafluorophosphate, 1-butyl-1-methylpyrrolidinium bromide, and triethylsulfonium bis(trifluoromethylsulfonyl)imide. The solvent may comprise a combination of solvents. In some embodiments, the solvent may comprise a buffer. A solvent comprising a buffer is referred to as a buffered solution, in some aspects. A buffered solution resists changes in pH when small amounts of acid or alkali are added. The buffer may be selected based upon the pH value of the resulting buffered solution. In some embodiments, the buffered solution pH is between about 4.0 and about 10.0. A list of exemplary buffers includes, but is not limited to, sodium borate/boric acid buffer, citrate/trisodium citrate buffer, monopotassium phosphate/dipotassium phosphate buffer, Dimethylarsinic acid (cacodylic acid) buffer, veronal-acetate buffer, s-Collidine buffer, 3-[N-Tris(hydroxymethyl)methylamino]-2-hydroxypropanesulfonic acid (TAPSO) buffer, 2-[4-(2-hydroxyethyl)piperazin-1-yl]ethanesulfonic acid (HEPES) buffer, 2-[[1,3-dihydroxy-2-(hydroxymethyl)propan-2-yl]amino]ethanesulfonic acid (TES) buffer, 3-Morpholinopropane-1-sulfonic acid (MOPS) buffer, 1,4-Piperazinediethanesulfonic acid (PIPES) buffer, and 2-(N-morpholino)ethanesulfonic acid (MES) buffer. Buffer concentrations may range from about 10 mM to about 100 Mm. Buffer concentrations may be about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, or 10000 mM (and any range derivable therein.

In specific embodiments, any of the buffers disclosed in this paragraph is specifically excluded as part of the embodiment.

In some aspects, a polar and polarizable molecule comprises a compound of the structure

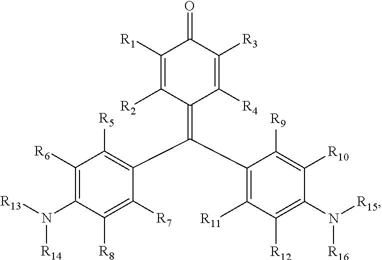

wherein R₁, R₂, R₃, R₄, R₅, R₆, R₇, R₈, R₉, R₁₀, R₁₁, R₁₂, R₁₃, R₁₄, R₁₅, and R₁₆ are each independently selected from hydrogen, alkyl, acyl, haloalky, hydroxyl, alkoxy, haloalkoxy, amino, nitro, mercapto, cyano, silyl, alkylsilyl, alkenyl, alkynyl, aryl, aralkyl, alkenoxy, alkynoxy, aryloxy, acyloxy, alkylamino, alkenylamino, alkynylamino, arylamino, amido, alkylthio, alkenylthio, alkynylthio, and arylthio.

In some embodiments, a polar and polarizable molecule comprises a compound of the structure

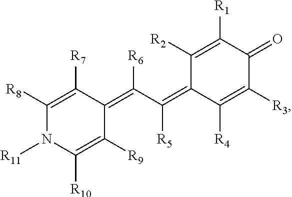

wherein R₁, R₂, R₃, R₄, R₅, R₆, R₇, R₈, R₉, R₁₀, and R₁₁ are each independently selected from hydrogen, alkyl, acyl, haloalky, hydroxyl, alkoxy, haloalkoxy, amino, nitro, mercapto, cyano, silyl, alkylsilyl, alkenyl, alkynyl, aryl, aralkyl, alkenoxy, alkynoxy, aryloxy, acyloxy, alkylamino, alkenylamino, alkynylamino, arylamino, amido, alkylthio, alkenylthio, alkynylthio, and arylthio.

In some embodiments, a polar and polarizable molecule is used to prepare a dye solution. In some embodiments, a polar and polarizable molecule is used to prepare a dye solution having a concentration of from about 1 μM to about 500 μM. The polar and polarizable molecule may be used to prepare a dye solution having a concentration of from about 10 μM to about 250 μM, in some aspects. The polar and polarizable molecule solution may be prepared in a concentration such that the polar and polarizable molecule dye solution provides an absorbance of c. 1.0 at its absorbance maxima. In various embodiments, concentrations may be about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, or 10000 μM (and any range derivable therein).

Light may interfere with polar and polarizable molecule-potency interaction and affect spectral measurement. Therefore, in some embodiments, a homeopathic potency solution, dye solution, reference solution, and/or sample solution is prepared in the absence of light. A homeopathic potency solution, dye solution, reference solution, and/or sample solution may be prepared in the absence of light with a wavelength of greater than 350 nm, in some aspects. A homeopathic potency solution, dye solution, reference solution, and/or sample solution may be kept in the dark from the time they are prepared until the time of spectral measurement thereof. In some embodiments, light with a wavelength of greater than 350 nm is prevented from reaching a reference solution (polar and polarizable molecule or dye solution) and sample solution from the time they are prepared until the time of spectral measurement thereof.

In some aspects, the measuring step comprises obtaining a plurality of sample solution and reference solution spectra at time intervals for up to 20 days, but usually up to 24 hours. In some embodiments, time interval may range from one minute to one hour. In some aspects, the step of obtaining a spectrum comprises measuring an absorbance spectrum. In other aspects, the step of obtaining a spectrum comprises measuring a fluorescence spectrum. In some embodiments, the sample solution has a volume of about 100 μL to 10 mL. In further embodiments, the sample solution has a volume of about 300 μL to 3 mL. In some embodiments, volumes may be about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, or 10000 μl or ml (and any range derivable therein.

In some embodiments, a spectrophotometer is used to obtain the sample and reference solution spectra. In some aspects, the sample and/or reference solution is kept in a quartz cuvette during measuring. In other aspects, the sample and/or reference solution is kept in a polystyrene, glass or UV cuvette during measuring. In some embodiments, the cuvettes may be re-used.

In some embodiments, the maximum value of the difference spectrum comprises the maximum of all difference spectra obtained over all time interval measurements. In further embodiments, a plot of the maximum of each difference spectrum vs. time is employed to determine a maximum rate of change of difference spectra.

In some embodiments, the polar and polarizable molecule is a solvatochromic dye selected from the group consisting of ET33, ET30, BM, BDN, BDF, NR, PB, DCM, PRODAN, DCVJ, stilbazolium dyes, coumarin dyes, ketocyanine dyes, analogs of Reichardt's dye, and merocyanine dyes or any other dye having electron donor and acceptor moieties linked through an electron-delocalized system in which the dye's dipole moment in the ground electronic state is considerably different from that in the excited state. In other embodiments, any of the polar and polarizable molecules is excluded in methods disclosed herein.

In some embodiments incubation of polar and polarizable molecule solutions with controls or homeopathic potencies are carried out in the dark and measurements made at time intervals for up to 20 days, but commonly for up to 24 hours. In some embodiments, the solution is maintained or measured under those conditions for the following or at least or at most for the following: 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months (or any range derivable therein).

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Structures of ET33 (2,6-Dichloro-4-(2,4,6-triphenyl-pyridinium-1-yl)-phenolate); ET30 (2,6-Diphenyl-4-(2,4,6-triphenyl-pyrinidium-1-yl)-phenolate); BM (4-[(E)-2-(1-methylpyridinium-4-yl)ethenyl]phenolate/Brooker's merocyanine) BDN ((4-(Bis-(4-(dimethylamino)phenyl)methylene)-1(4H)-naphthalenone); BDF (4-[bis[4-(dimethylamino) phenyl]methylene]-2,5-cyclohexadien-1-one); NR (9-diethylamino-5H-benzo[a]phenoxazime-5-one/Nile Red); PB (N,N-dimethylindoaniline/Phenol Blue); 7-(dimethylamino)-3H-phenothiazin-3-one (methylene violet, MV); 6-amino-2-naphthoic acid (6ANA); 6-amino-2-anthracenic acid (6AAA); (E)-2-cyano-3-(5-(4-(diphenylamino)phenyl)thiophen-2-yl)acrylic acid (L), and (E)-2-cyano-3-(5-(4-(diphenylamino)styryl)thiophen-2-yl)acrylic acid (D5). ET33, ET30 and BM are negatively solvatochromic dyes. D5, L1, BDN, BDF, NR and PB are positively solvatochromic dyes. 6ANA and 6AAA are halochromic dyes.

FIG. 2—Diagram showing the structure of ground and excited states in negatively and positively solvatochromic dyes. For the positively solvatochromic dyes used in this study electron donating groups (D) are of the form ═N— and electron accepting groups (A) are of the form ═C═O. For the negatively solvatochromic dyes used in this study electron donating groups are of the form —O⁻ and acceptor groups are of the form ≡N⁺—.

FIG. 3—Schematic representation of H-aggregates (A) and J-aggregates (B) in solution.

FIG. 4—Difference spectrum of ET33 in ethanol with 50 μl of control added to the reference cuvette and 50 μl of glycerol 50 M added to the sample cuvette to make a total volume of 3 ml in each cuvette. ET33 is at a concentration of 245 μM. The difference spectrum consists of the difference between the sample spectrum and the reference spectrum. Difference spectrum shown is that at t=120 min after mixing. UV cuvettes used. Insert shows the change in OD442 nm over time.

FIG. 5—Absorbance loss over time at 472 nm for 245 μM ET33 in ethanol with 70 μM SrCl₂ and 50 μl control (upper curve) or 70 μM SrCl₂ and 50 μl glycerol 50M (lower curve). Assays carried out in both PS and UV cuvettes. N=20; error bars are to first standard deviation; p<0.0001, indicating high statistical significance.

FIG. 6—Difference spectrum of ET30 in tert-butyl alcohol with 50 μl of control added to the reference cuvette and 50 μl of glycerol 50 M added to the sample cuvette to make a total volume of 3 ml in each cuvette (see Materials and methods). ET30 is at a concentration of 245 μM. Difference spectrum consists of the sample spectrum minus the reference spectrum. Difference spectrum shown is that at t=120 min after mixing. UV cuvettes used.

FIG. 7—Difference spectrum of BDN in ethanol with 50 μl of control added to the reference cuvette and 50 μl of glycerol 50 M added to the sample cuvette to make a total volume of 3 ml in each cuvette (see Materials and methods). BDN is at a concentration of 80 μM. Difference spectrum consists of the sample spectrum minus the reference spectrum. Difference spectrum shown is that at t=30 min after mixing. UV cuvettes used.

FIG. 8—Absorbance gain over time at 615 nm for 80 μM BDN in ethanol with 70 μM SrCl₂ and 50 μl control (upper curve) or 70 μM SrCl₂ and 50 μl glycerol 50 M (lower curve). Assays carried out in both PS and UV cuvettes. N=20; error bars are to first standard deviation; p<0.0001, indicating high statistical significance.

FIG. 9—Difference spectrum of BM in tert-butyl alcohol with 50 μl of control added to the reference cuvette and 50 μl of glycerol 50M added to the sample cuvette to make a total volume of 3 ml in each cuvette (see Materials and methods). BM is at a concentration of 125 μM. Difference spectrum consists of the sample spectrum minus the reference spectrum. Difference spectrum shown is that at t=40 min after mixing. UV cuvettes used.

FIG. 10—Combined difference spectra (n=20) of ET30 in tert-butyl alcohol with 50 μl of control added to the reference cuvette and 50 μl of glycerol 50 M added to the sample cuvette to make a total volume of 3 ml in each cuvette. ET30 is at a concentration of 245 μM. Difference spectrum consists of the sample spectrum minus the reference spectrum. UV cuvettes used. Error bars are to the first standard deviation. Control ‘difference’ spectra (n=20) where 50 μl of control are added to both cuvettes display no discernible difference across the spectrum from 350 to 800 nm (i.e., 0.000±0.000) giving a p value of <0.0001, indicating high statistical significance.

FIG. 11—Difference spectrum of BDF in ROW with 50 μl of control added to the reference cuvette and 50 μl of glycerol 50M added to the sample cuvette to make a total volume of 3 ml in each cuvette. BDF is at a concentration of 15 μM. Difference spectrum consists of the sample spectrum minus the reference spectrum. Difference spectrum shown is that at t=100 min after mixing. UV cuvettes used.

FIG. 12—A table demonstrating the effects of potencies and light on dyes. Light inhibits potencies from acting on the dyes. The results suggest investigations should be carried out as much as possible in the dark.

FIG. 13—A graph correlating the change in dye spectra as a function of polar and polarizable molecule dipole moment. The results indicate that the degree of interaction between potency and dye is dependent upon the magnitude of the dipole moment of the dye in question. Furthermore, results are seen with halochromic dyes (pH-sensitive) and solvatochromic dyes, both of which are polar and polarizable molecules.

### DESCRIPTION OF ILLUSTRATIVE EMBODIMENTS

### I. Introduction

A plausible and testable hypothesis for the mode of action of homeopathy and, by implication, an understanding of the physico-chemical nature of homeopathic potencies, would profoundly enhance homeopathy, both as an area of legitimate scientific study and as an effective medical approach. Research at the molecular level has the advantage over other approaches in that it can ask the kinds of searching and detailed questions necessary to arrive at fully testable hypotheses as to the modus operandi of homeopathy.

With this view in mind a program of investigation aimed at developing well-defined chemical systems capable of detecting consistent and replicable effects of serially diluted and agitated solutions was initiated. Specifically, a simple chemical system utilizing environment-sensitive polar and polarizable molecules has been developed. Polar and polarizable molecules are sensitive to, and can be used to follow, a range of solution dynamics through changes in their absorbance spectra which may occur in the visible portion of the electromagnetic spectrum.

The system described below demonstrates not only that homeopathic potencies have in vitro effects which can be measured, but also because the system is both simple and versatile, very specific questions can be asked about what molecular effects potencies are having in solution and what their ultimate nature might be.

Whilst a range of chemical and physical systems have been employed in the past in the study of homeopathic medicines, including UV-spectroscopy (Wolf, et al., 2011), nuclear magnetic resonance spectroscopy (Aabel, et al., 2001), thermoluminescence (Rey, 2003), high voltage plasma visualisation (Assumpcao, 2008), solution conductivity (Elia, et al., 2004) and micro-calorimetry (Belon, et al., 2008), together with theoretical studies (Bell, 2008), little consensus has emerged as to the nature of the homeopathic stimulus. Results suggest potencies may be electromagnetic in nature (Montagnier, 2009), or that they may involve and exploit the intrinsic ability of water to form complex hydrogen bonding networks (Yin Lo, et al., 2009). They may have their origins in quantum electrodynamics (Marchettini, et al., 2010), quantum entanglement (Milgrom, 2006), complexity theory (Bellavite, 2003) or stochastic resonance (Torres, 1996).

The present approach has grown out of a recognition that a number of criteria need to be fulfilled if homeopathic potencies are to be studied in a way that provides results that are (i) significantly above background noise and (ii) allows the development of systems that can be manipulated to reveal the effect of one variable at a time. The criteria are essentially two-fold. The first involves the stability of homeopathic potencies. The destruction of potencies is important if one is to avoid cross-contamination where glassware and other container materials are re-used. For this reason the current study has employed disposable containers in situations where cross-contamination is a potential problem.

The second group of criteria revolves around the issue of the control of variables. Ideally, any detection system should be one in which the variables involved can be addressed individually. In this way, specific questions can be asked and specific answers obtained. A well-defined and simple detection system is therefore highly desirable, especially if the system is capable of providing different types of information.

With these criteria in mind, a detection system involving polar and polarizable molecules has been developed. In brief, changes have been found to occur in the absorbance spectra of these molecules in the presence of homeopathic potencies. In turn it has then been possible to make certain inferences as to the specific action of potencies in a solution.

A system in which homeopathic a potency is added to a solution of a polar and polarizable molecule is simple, versatile, and involves a very small number of components and operational steps. In addition, the system is sensitive to changes in a wide range of solution dynamics including solvent polarity, solvent pH, solvent-solute binding patterns, and supramolecular interactions between solute molecules.

### II. Homeopathic Potency

In some embodiments, the method is used to detect, measure or quantify a homeopathic potency or obtain a characteristic of a homeopathic solution.

In homeopathy, homeopathic dilution (known by practitioners as “dynamisation” or “potentisation”) is a process in which a substance is diluted with alcohol or distilled water and then vigorously shaken in a process called “succussion”. For example, insoluble solids, such as quartz and oyster shell, may be diluted by grinding them with lactose (trituration).

Several potency scales are in use in homeopathy. The centesimal or “C scale” dilutes a substance by a factor of 100 at each stage. A 2C dilution requires a substance to be diluted to one part in one hundred, and then some of that diluted solution diluted by a further factor of one hundred. This works out to one part of the original substance in 10,000 parts of the solution. A 6C dilution repeats this process six times, ending up with the original material diluted by a factor of 100⁶=10¹². Higher dilutions follow the same pattern. In homeopathy, a solution that is more dilute is described as having a higher potency, and more dilute substances are considered by homeopaths to be stronger and deeper-acting. The end product is often so diluted that it is indistinguishable from the diluent (pure water, sugar or alcohol). The greatest dilution that is reasonably likely to contain one molecule of the original substance is 12C, if starting from 1 mole of original substance.

There is too the continued flow mode of dilution that is measured on mass flow controller (MFC) based dilution systems.

Some homeopaths developed a decimal scale (D or X), diluting the substance to ten times its original volume each stage. The D or X scale dilution is therefore half that of the same value of the C scale; for example, “12X” is the same level of dilution as “6C”. In other examples, there is a quintamillesimal (Q) or LM scale, diluting the drug 1 part in 50,000 parts of diluent.[9] A given dilution on the Q scale is roughly 2.35 times its designation on the C scale. For example a preparation described as “20Q” has about the same concentration as one described with “47C” g.

Potencies of 1000c and above are usually labelled with Roman numeral M and with the centesimal ‘c’ indicator implied (since all such high potencies are centesimal dilutions): 1M=1000c; 10M=10,000c; CM=100,000c; LM (which would indicate 50,000c) is typically not used due to confusion with the LM potency scale.

In certain aspects, the methods may involve the characterization of certain homeopathic medicines. Many remedies at different potencies have been investigated during the course of the present study and the results obtained are broadly comparable to the results presented in Example 1.

### III. Solvatochromic Dyes

In certain aspects, a solvatochromic dye may be employed as a polar and polarizable molecule, for example, in detecting, measuring, and/or quantifying homeopathic potency.

Solvatochromic dyes, or solvatochromic compounds, include compounds having spectroscopic characteristics (e.g., absorption, emission, fluorescence, phosphorescence) in the ultraviolet/visible/near-infrared spectrum that are influenced by the surrounding medium. Examples of solvatochromic dyes suitable for use with the disclosed methods include any known solvatochromic dyes. Solvatochromic dyes have been extensively reviewed in, for example, Reichardt, Chemical Reviews, 94:2319-2358 (1994); Reichardt et al. Pure and Applied Chemistry 65(12):2593-601 (1993); and Buncel and Rajagopal, Accounts of Chemical Research, 23(7):226-31 (1990), all of which are incorporated herein by reference in their entirety.

Characteristics of solvatochromic compounds include positive or negative solvatochromism, which corresponds to the bathochromic and hypsochromic shifts, respectively of the emission band with increasing solvent polarity. In addition to the solvent-induced spectral shifts of absorbance peaks, some compounds exhibit the solvent-dependent fluorescence emission peaks. Examples of such solvatochromic compounds include Nile Red and Brooker's merocyanine.

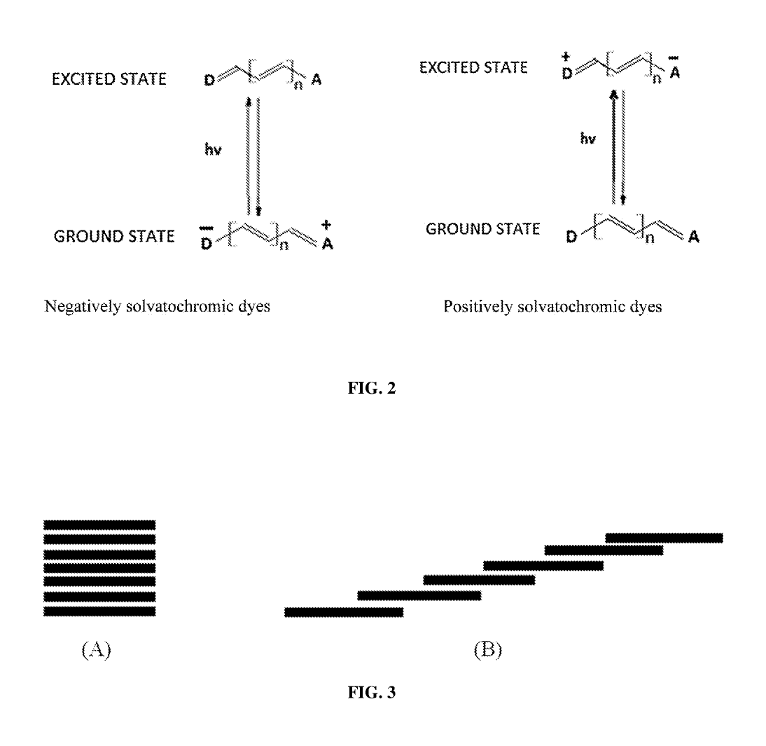

Solvatochromic dyes can be characterized by possessing an electron donating group and an electron accepting group with an electron delocalized system in-between (Reichardt, et al., 2008). For negatively solvatochromic dyes the ground or resting state is zwitterionic with a formal charge at either end of the molecule. On absorption of light an electron travels from one end of the molecule to the other to form an excited polar, but uncharged, state (FIG. 2). Whilst the lifetime of the excited state is of the order of picoseconds this rapid electron oscillation occurs constantly under the influence of absorbed light. Importantly the wavelength of the absorbed light is influenced by the environment in which the dye is placed. Conversely, for positively solvatochromic dyes the ground or resting state is uncharged. On absorption of light an electron travels from one end of the molecule to the other to form an equally short-lived charged excited state (FIG. 2). Again the wavelength of the light absorbed is dependent upon the nature of the environment in which the dye is placed. Negatively and positively solvatochromic dyes behave differently and complementarily to each other in a number of ways. These are important in relation to the results obtained with potencies reported below.

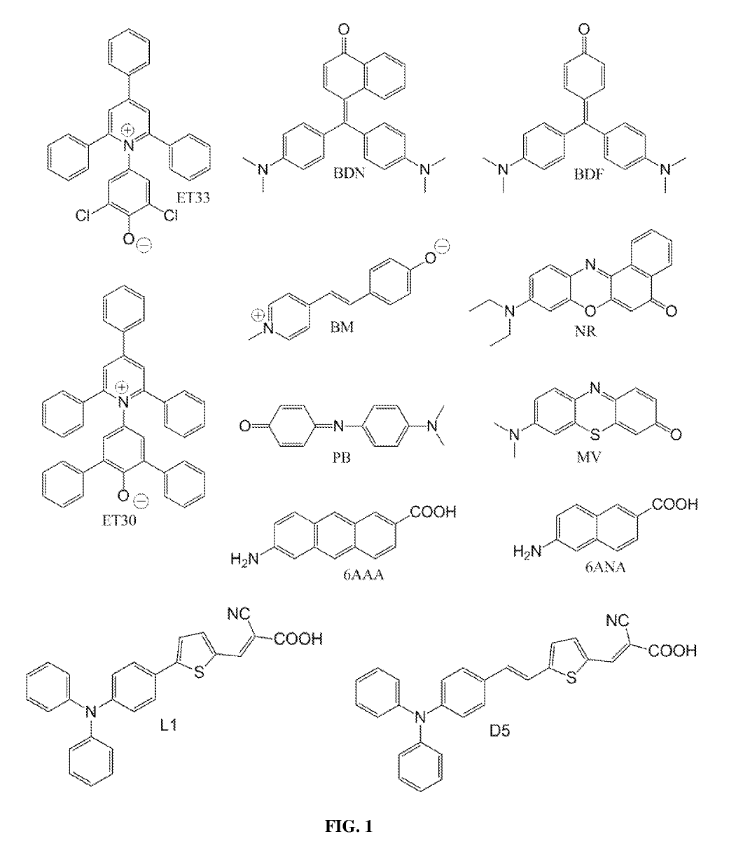

Negatively solvatochromic dyes absorb at longer and longer wavelengths (bathochromically shifted) as solvent polarity decreases. For example ET30 (FIG. 1) absorbs at 450 nm in water, 550 nm in ethanol and 650 nm in tert-butyl alcohol (Reichardt, 1994). In addition these dyes tend to increasingly aggregate as solvent polarity decreases producing aggregates that are bathochromically shifted with respect to monomer (unaggregated material), a phenomenon known as aggregachromism (Banfield & Hutchings, 2010). These bathochromically shifted species take the form of ‘steps’ in solution and are known as J-aggregates (Wurthner, et al., 2002) (FIG. 3).

Conversely, positively solvatochromic dyes absorb at shorter and shorter wavelengths (hypsochromically shifted) as solvent polarity decreases. For example BDN (FIG. 1) absorbs at 614 nm in water, 564 nm in ethanol and 537 nm in tert-butyl alcohol. These dyes tend to increasingly aggregate as solvent polarity decreases producing aggregates that are hypsochromically shifted with respect to monomer (Eisfield 2006), in contrast to that seen with negatively solvatochromic dyes. These hypsochromically shifted species take the form of ‘stacks’ in solution and are known as H-aggregates (Mishra, et al., 2000) (FIG. 3).

Dyes ET33 (2,6-dichloro-4-(2,4,6-triphenyl-N-pyridino)-phenolate), ET30 ((2,4,6-triphenyl-1-pyridinio)-1-phenolate) and BM (Brooker's merocyanine) produce J-aggregates in solution; dyes BDN, BDF, NR, and PB produce H-aggregates (Balzani, et al., 2014).

In addition, dyes ET30, ET33 BDF, and BDN bind divalent cations (Reichardt, 1992) to produce optical changes which can also be utilized to demonstrate effects of homeopathic potencies.

Additional examples of solvatochromic compounds include, but are not limited to 4-dicyanmethylene-2-methyl-6-(p-dimethylaminostyryl)-4H-pyran (DCM); 6-propionyl-2-(dimethylamino)naphthalene (PRODAN); 4-(dicyanovinyl)julolidine (DCVJ); stilbazolium dyes; coumarin dyes; ketocyanine dyes; analogs of Reichardt's dyes; merocyanine dyes, including merocyanine 540; N,N-dimethyl-4-nitroaniline (NDMNA) and the like. Other solvatochromic dyes include, but are not limited to all compounds having electron donor and acceptor moieties linked through an electron delocalized system in which the dipole moment in the electronic ground state is considerably different from that in the excited state.

### IV. Halochromic Dyes

In certain aspects, a halochromic dye may be employed as a polar and polarizable molecule, for example, in detecting, measuring, and/or quantifying homeopathic potency.

Halochromic dyes change color as the pH of the solvent in which they are dissolved or mixed changes. In some aspects, the molecular structure of a halochromic dye changes in response to a change in pH, as in the case of phenolphthalein. pH indicator molecules may be utilized to demonstrate the effects of homeopathic potencies.

As used herein, the term “amino” means —NH₂; the term “nitro” means —NO₂; the term “halo” or “halogen” designates —F, —Cl, —Br or —I; the term “mercapto” means —SH; the term “cyano” means —CN; the term “azido” means —N₃; the term “silyl” means —SiH₃, and the term “hydroxy” means —OH. In certain embodiments, a halogen may be —Br or —I.

The term “alkyl” includes straight-chain alkyl, branched-chain alkyl, cycloalkyl (alicyclic), cyclic alkyl, heteroatom-unsubstituted alkyl, heteroatom-substituted alkyl, heteroatom-unsubstituted Cₙ-alkyl, and heteroatom-substituted Cₙ-alkyl. In certain embodiments, lower alkyls are contemplated. The term “lower alkyl” refers to alkyls of 1-6 carbon atoms (that is, 1, 2, 3, 4, 5 or 6 carbon atoms). The term “heteroatom-unsubstituted Cₙ-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups, —CH₃ (Me), —CH₂CH₃ (Et), —CH₂CH₂CH₃ (n-Pr), —CH(CH₃)₂(iso-Pr), —CH(CH₂)₂(cyclopropyl), —CH₂CH₂CH₂CH₃ (n-Bu), —CH(CH₃)CH₂CH₃ (sec-butyl), —CH₂CH(CH₃)₂(iso-butyl), —C(CH₃)₃(tert-butyl), —CH₂C(CH₃)₃(neo-pentyl), cyclobutyl, cyclopentyl, and cyclohexyl, are all non-limiting examples of heteroatom-unsubstituted alkyl groups. The term “heteroatom-substituted Cₙ-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all non-limiting examples of heteroatom-substituted alkyl groups: trifluoromethyl, —CH₂F, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The term “alkenyl” includes straight-chain alkenyl, branched-chain alkenyl, cycloalkenyl, cyclic alkenyl, heteroatom-unsubstituted alkenyl, heteroatom-substituted alkenyl, heteroatom-unsubstituted Cₙ-alkenyl, and heteroatom-substituted Cₙ-alkenyl. In certain embodiments, lower alkenyls are contemplated. The term “lower alkenyl” refers to alkenyls of 1-6 carbon atoms (that is, 1, 2, 3, 4, 5 or 6 carbon atoms). The term “heteroatom-unsubstituted Cₙ-alkenyl” refers to a radical, having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C₂-C₁₀-alkenyl has 2 to 10 carbon atoms. Heteroatom-unsubstituted alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “heteroatom-substituted Cₙ-alkenyl” refers to a radical, having a single nonaromatic carbon atom as the point of attachment and at least one nonaromatic carbon-carbon double bond, but no carbon-carbon triple bonds, further having a linear or branched, cyclic or acyclic structure, further having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkenyl has 2 to 10 carbon atoms. The groups, —CH═CHF, —CH═CHCl and —CH═CHBr, are non-limiting examples of heteroatom-substituted alkenyl groups.

The term “aryl” includes heteroatom-unsubstituted aryl, heteroatom-substituted aryl, heteroatom-unsubstituted Cₙ-aryl, heteroatom-substituted Cₙ-aryl, heteroaryl, heterocyclic aryl groups, carbocyclic aryl groups, biaryl groups, and single-valent radicals derived from polycyclic fused hydrocarbons (PAHs). The term “heteroatom-unsubstituted Cₙ-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C₆-C₁₀-aryl has 6 to 10 carbon atoms. Non-limiting examples of heteroatom-unsubstituted aryl groups include phenyl (Ph), methylphenyl, (dimethyl)phenyl, —C₆H₄CH₂CH₃, —C₆H₄CH₂CH₂CH₃, —C₆H₄CH(CH₃)₂, —C₆H₄CH(CH₂)₂, —C₆H₃(CH₃)CH₂CH₃, —C₆H₄CH═CH₂, —C₆H₄CH═CHCH₃, —C₆H₄C≡CH, —C₆H₄C≡CCH₃, naphthyl, and the radical derived from biphenyl. The term “heteroatom-substituted Cₙ-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. Non-limiting examples of heteroatom-substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, —C₆H₄CON(CH₃)₂, furanyl, thienyl, pyridyl, pyrrolyl, pyrimidyl, pyrazinyl, quinolyl, indolyl, and imidazoyl. In certain embodiments, heteroatom-substituted aryl groups are contemplated. In certain embodiments, heteroatom-unsubstituted aryl groups are contemplate. In certain embodiments, an aryl group may be mono-, di-, tri-, tetra- or penta-substituted with one or more heteroatom-containing substitutents.

The term “aralkyl” includes heteroatom-unsubstituted aralkyl, heteroatom-substituted aralkyl, heteroatom-unsubstituted Cₙ-aralkyl, heteroatom-substituted Cₙ-aralkyl, heteroaralkyl, and heterocyclic aralkyl groups. In certain embodiments, lower aralkyls are contemplated. The term “lower aralkyl” refers to aralkyls of 7-12 carbon atoms (that is, 7, 8, 9, 10, 11 or 12 carbon atoms). The term “heteroatom-unsubstituted Cₙ-aralkyl” refers to a radical, having a single saturated carbon atom as the point of attachment, further having a total of n carbon atoms, wherein at least 6 of the carbon atoms form an aromatic ring structure containing only carbon atoms, 7 or more hydrogen atoms, and no heteroatoms. For example, a heteroatom-unsubstituted C₇-C₁₀-aralkyl has 7 to 10 carbon atoms. Non-limiting examples of heteroatom-unsubstituted aralkyls are: phenylmethyl (benzyl, Bn) and phenylethyl. The term “heteroatom-substituted Cₙ-aralkyl” refers to a radical, having a single saturated carbon atom as the point of attachment, further having a total of n carbon atoms, 0, 1, or more than one hydrogen atom, and at least one heteroatom, wherein at least one of the carbon atoms is incorporated an aromatic ring structures, further wherein each heteroatom is independently selected from the group consisting of N, O, F, Cl, Br, I, Si, P, and S. For example, a heteroatom-substituted C₂-C₁₀-heteroaralkyl has 2 to 10 carbon atoms.

The term “acyl” includes straight-chain acyl, branched-chain acyl, cycloacyl, cyclic acyl, heteroatom-unsubstituted acyl, heteroatom-substituted acyl, heteroatom-unsubstituted Cₙ-acyl, heteroatom-substituted Cₙ-acyl, alkylcarbonyl, alkoxycarbonyl and aminocarbonyl groups. In certain embodiments, lower acyls are contemplated. The term “lower acyl” refers to acyls of 1-6 carbon atoms (that is, 1, 2, 3, 4, 5 or 6 carbon atoms). The term “heteroatom-unsubstituted Cₙ-acyl” refers to a radical, having a single carbon atom of a carbonyl group as the point of attachment, further having a linear or branched, cyclic or acyclic structure, further having a total of n carbon atoms, 1 or more hydrogen atoms, a total of one oxygen atom, and no additional heteroatoms. For example, a heteroatom-unsubstituted C₁-C₁₀-acyl has 1 to 10 carbon atoms. The groups, —CHO, —C(O)CH₃, —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, and —COC₆H₃(CH₃)₂, are non-limiting examples of heteroatom-unsubstituted acyl groups. The term “heteroatom-substituted Cₙ-acyl” refers to a radical, having a single carbon atom as the point of attachment, the carbon atom being part of a carbonyl group, further having a linear or branched, cyclic or acyclic structure, further having a total of n carbon atoms, 0, 1, or more than one hydrogen atom, at least one additional heteroatom, in addition to the oxygen of the carbonyl group, wherein each additional heteroatom is independently selected from the group consisting of N, O, F, Cl, Br, I, Si, P, and S. For example, a heteroatom-substituted C₁-C₁₀-acyl has 1 to 10 carbon atoms. The groups, —C(O)CH₂CF₃, —CO₂H, —CO₂—, —CO₂CH₃, —CO₂CH₂CH₃, —CO₂CH₂CH₂CH₃, —CO₂CH(CH₃)₂, —CO₂CH(CH₂)₂, —C(O)NH₂ (carbamoyl), —C(O)NHCH₃, —C(O)NHCH₂CH₃, —CONHCH(CH₃)₂, —CONHCH(CH₂)₂, —CON(CH₃)₂, and —CONHCH₂CF₃, are non-limiting examples of heteroatom-substituted acyl groups.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted Cₙ-alkoxy, and heteroatom-substituted C-alkoxy. In certain embodiments, lower alkoxys are contemplated. The term “lower alkoxy” refers to alkoxys of 1-6 carbon atoms (that is, 1, 2, 3, 4, 5 or 6 carbon atoms). The term “heteroatom-unsubstituted Cₙ-alkoxy” refers to a group, having the structure —OR, in which R is a heteroatom-unsubstituted Cₙ-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂. The term “heteroatom-substituted Cₙ-alkoxy” refers to a group, having the structure —OR, in which R is a heteroatom-substituted Cₙ-alkyl, as that term is defined above. For example, —OCH₂CF₃ is a heteroatom-substituted alkoxy group.

The term “alkenyloxy” includes straight-chain alkenyloxy, branched-chain alkenyloxy, cycloalkenyloxy, cyclic alkenyloxy, heteroatom-unsubstituted alkenyloxy, heteroatom-substituted alkenyloxy, heteroatom-unsubstituted Cₙ-alkenyloxy, and heteroatom-substituted Cₙ-alkenyloxy. The term “heteroatom-unsubstituted Cₙ-alkenyloxy” refers to a group, having the structure —OR, in which R is a heteroatom-unsubstituted Cₙ-alkenyl, as that term is defined above. The term “heteroatom-substituted Cₙ-alkenyloxy” refers to a group, having the structure —OR, in which R is a heteroatom-substituted Cₙ-alkenyl, as that term is defined above.

The term “alkynyloxy” includes straight-chain alkynyloxy, branched-chain alkynyloxy, cycloalkynyloxy, cyclic alkynyloxy, heteroatom-unsubstituted alkynyloxy, heteroatom-substituted alkynyloxy, heteroatom-unsubstituted Cₙ-alkynyloxy, and heteroatom-substituted Cₙ-alkynyloxy. The term “heteroatom-unsubstituted Cₙ-alkynyloxy” refers to a group, having the structure —OR, in which R is a heteroatom-unsubstituted Cₙ-alkynyl, as that term is defined above. The term “heteroatom-substituted Cₙ-alkynyloxy” refers to a group, having the structure —OR, in which R is a heteroatom-substituted Cₙ-alkynyl, as that term is defined above.

The term “aryloxy” includes heteroatom-unsubstituted aryloxy, heteroatom-substituted aryloxy, heteroatom-unsubstituted Cₙ-aryloxy, heteroatom-substituted Cₙ-aryloxy, heteroaryloxy, and heterocyclic aryloxy groups. The term “heteroatom-unsubstituted Cₙ-aryloxy” refers to a group, having the structure —OAr, in which Ar is a heteroatom-unsubstituted Cₙ-aryl, as that term is defined above. A non-limiting example of a heteroatom-unsubstituted aryloxy group is —OC₆H₅. The term “heteroatom-substituted Cₙ-aryloxy” refers to a group, having the structure —OAr, in which Ar is a heteroatom-substituted Cₙ-aryl, as that term is defined above.

The term “aralkyloxy” includes heteroatom-unsubstituted aralkyloxy, heteroatom-substituted aralkyloxy, heteroatom-unsubstituted Cₙ-aralkyloxy, heteroatom-substituted Cₙ-aralkyloxy, heteroaralkyloxy, and heterocyclic aralkyloxy groups. The term “heteroatom-unsubstituted Cₙ-aralkyloxy” refers to a group, having the structure —OAr, in which Ar is a heteroatom-unsubstituted Cₙ-aralkyl, as that term is defined above. The term “heteroatom-substituted Cₙ-aralkyloxy” refers to a group, having the structure —OAr, in which Ar is a heteroatom-substituted Cₙ-aralkyl, as that term is defined above.

The term “acyloxy” includes straight-chain acyloxy, branched-chain acyloxy, cycloacyloxy, cyclic acyloxy, heteroatom-unsubstituted acyloxy, heteroatom-substituted acyloxy, heteroatom-unsubstituted Cₙ-acyloxy, heteroatom-substituted Cₙ-acyloxy, alkylcarbonyloxy, arylcarbonyloxy, alkoxycarbonyloxy, aryloxycarbonyloxy, and carboxylate groups. The term “heteroatom-unsubstituted Cₙ-acyloxy” refers to a group, having the structure —OAc, in which Ac is a heteroatom-unsubstituted Cₙ-acyl, as that term is defined above. For example, —OC(O)CH₃ is a non-limiting example of a heteroatom-unsubstituted acyloxy group. The term “heteroatom-substituted Cₙ-acyloxy” refers to a group, having the structure —OAc, in which Ac is a heteroatom-substituted Cₙ-acyl, as that term is defined above. For example, —OC(O)OCH₃ and —OC(O)NHCH₃ are non-limiting examples of heteroatom-unsubstituted acyloxy groups.

The term “alkylamino” includes straight-chain alkylamino, branched-chain alkylamino, cycloalkylamino, cyclic alkylamino, heteroatom-unsubstituted alkylamino, heteroatom-substituted alkylamino, heteroatom-unsubstituted Cₙ-alkylamino, and heteroatom-substituted Cₙ-alkylamino. The term “heteroatom-unsubstituted Cₙ-alkylamino” refers to a radical, having a single nitrogen atom as the point of attachment, further having one or two saturated carbon atoms attached to the nitrogen atom, further having a linear or branched, cyclic or acyclic structure, containing a total of n carbon atoms, all of which are nonaromatic, 4 or more hydrogen atoms, a total of 1 nitrogen atom, and no additional heteroatoms. For example, a heteroatom-unsubstituted C₁-C₁₀-alkylamino has 1 to 10 carbon atoms. The term “heteroatom-unsubstituted Cₙ-alkylamino” includes groups, having the structure —NHR, in which R is a heteroatom-unsubstituted Cₙ-alkyl, as that term is defined above. A heteroatom-unsubstituted alkylamino group would include —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —N(CH₃)₂, —N(CH₃)CH₂CH₃, —N(CH₂CH₃)₂, N-pyrrolidinyl, and N-piperidinyl. The term “heteroatom-substituted Cₙ-alkylamino” refers to a radical, having a single nitrogen atom as the point of attachment, further having one or two saturated carbon atoms attached to the nitrogen atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₁-C₁₀-alkylamino has 1 to 10 carbon atoms. The term “heteroatom-substituted Cₙ-alkylamino” includes groups, having the structure —NHR, in which R is a heteroatom-substituted Cₙ-alkyl, as that term is defined above.

The term “alkenylamino” includes straight-chain alkenylamino, branched-chain alkenylamino, cycloalkenylamino, cyclic alkenylamino, heteroatom-unsubstituted alkenylamino, heteroatom-substituted alkenylamino, heteroatom-unsubstituted Cₙ-alkenylamino, heteroatom-substituted Cₙ-alkenylamino, dialkenylamino, and alkyl(alkenyl)amino groups. The term “heteroatom-unsubstituted Cₙ-alkenylamino” refers to a radical, having a single nitrogen atom as the point of attachment, further having one or two carbon atoms attached to the nitrogen atom, further having a linear or branched, cyclic or acyclic structure, containing at least one nonaromatic carbon-carbon double bond, a total of n carbon atoms, 4 or more hydrogen atoms, a total of one nitrogen atom, and no additional heteroatoms. For example, a heteroatom-unsubstituted C₂-C₁₀-alkenylamino has 2 to 10 carbon atoms. The term “heteroatom-unsubstituted Cₙ-alkenylamino” includes groups, having the structure —NHR, in which R is a heteroatom-unsubstituted Cₙ-alkenyl, as that term is defined above. The term “heteroatom-substituted Cₙ-alkenylamino” refers to a radical, having a single nitrogen atom as the point of attachment and at least one nonaromatic carbon-carbon double bond, but no carbon-carbon triple bonds, further having one or two carbon atoms attached to the nitrogen atom, further having a linear or branched, cyclic or acyclic structure, further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkenylamino has 2 to 10 carbon atoms. The term “heteroatom-substituted Cₙ-alkenylamino” includes groups, having the structure —NHR, in which R is a heteroatom-substituted Cₙ-alkenyl, as that term is defined above.

The term “alkynylamino” includes straight-chain alkynylamino, branched-chain alkynylamino, cycloalkynylamino, cyclic alkynylamino, heteroatom-unsubstituted alkynylamino, heteroatom-substituted alkynylamino, heteroatom-unsubstituted Cₙ-alkynylamino, heteroatom-substituted Cₙ-alkynylamino, dialkynylamino, alkyl(alkynyl)amino, and alkenyl(alkynyl)amino groups. The term “heteroatom-unsubstituted Cₙ-alkynylamino” refers to a radical, having a single nitrogen atom as the point of attachment, further having one or two carbon atoms attached to the nitrogen atom, further having a linear or branched, cyclic or acyclic structure, containing at least one carbon-carbon triple bond, a total of n carbon atoms, at least one hydrogen atoms, a total of one nitrogen atom, and no additional heteroatoms. For example, a heteroatom-unsubstituted C₂-C₁₀-alkynylamino has 2 to 10 carbon atoms. The term “heteroatom-unsubstituted Cₙ-alkynylamino” includes groups, having the structure —NHR, in which R is a heteroatom-unsubstituted Cₙ-alkynyl, as that term is defined above. The term “heteroatom-substituted Cₙ-alkynylamino” refers to a radical, having a single nitrogen atom as the point of attachment, further having one or two carbon atoms attached to the nitrogen atom, further having at least one nonaromatic carbon-carbon triple bond, further having a linear or branched, cyclic or acyclic structure, and further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkynylamino has 2 to 10 carbon atoms. The term “heteroatom-substituted Cₙ-alkynylamino” includes groups, having the structure —NHR, in which R is a heteroatom-substituted Cₙ-alkynyl, as that term is defined above.

The term “arylamino” includes heteroatom-unsubstituted arylamino, heteroatom-substituted arylamino, heteroatom-unsubstituted Cₙ-arylamino, heteroatom-substituted Cₙ-arylamino, heteroarylamino, heterocyclic arylamino, and alkyl(aryl)amino groups. The term “heteroatom-unsubstituted Cₙ-arylamino” refers to a radical, having a single nitrogen atom as the point of attachment, further having at least one aromatic ring structure attached to the nitrogen atom, wherein the aromatic ring structure contains only carbon atoms, further having a total of n carbon atoms, 6 or more hydrogen atoms, a total of one nitrogen atom, and no additional heteroatoms. For example, a heteroatom-unsubstituted C₆-C₁₀-arylamino has 6 to 10 carbon atoms. The term “heteroatom-unsubstituted Cₙ-arylamino” includes groups, having the structure —NHR, in which R is a heteroatom-unsubstituted Cₙ-aryl, as that term is defined above. The term “heteroatom-substituted Cₙ-arylamino” refers to a radical, having a single nitrogen atom as the point of attachment, further having a total of n carbon atoms, at least one hydrogen atom, at least one additional heteroatoms, that is, in addition to the nitrogen atom at the point of attachment, wherein at least one of the carbon atoms is incorporated into one or more aromatic ring structures, further wherein each additional heteroatom is independently selected from the group consisting of N, O, F, Cl, Br, I, Si, P, and S. For example, a heteroatom-substituted C₆-C₁₀-arylamino has 6 to 10 carbon atoms. The term “heteroatom-substituted Cₙ-arylamino” includes groups, having the structure —NHR, in which R is a heteroatom-substituted Cₙ-aryl, as that term is defined above.

The term “aralkylamino” includes heteroatom-unsubstituted aralkylamino, heteroatom-substituted aralkylamino, heteroatom-unsubstituted Cₙ-aralkylamino, heteroatom-substituted Cₙ-aralkylamino, heteroaralkylamino, heterocyclic aralkylamino groups, and diaralkylamino groups. The term “heteroatom-unsubstituted Cₙ-aralkylamino” refers to a radical, having a single nitrogen atom as the point of attachment, further having one or two saturated carbon atoms attached to the nitrogen atom, further having a total of n carbon atoms, wherein at least 6 of the carbon atoms form an aromatic ring structure containing only carbon atoms, 8 or more hydrogen atoms, a total of one nitrogen atom, and no additional heteroatoms. For example, a heteroatom-unsubstituted C₇-C₁₀-aralkylamino has 7 to 10 carbon atoms. The term “heteroatom-unsubstituted Cₙ-aralkylamino” includes groups, having the structure —NHR, in which R is a heteroatom-unsubstituted Cₙ-aralkyl, as that term is defined above. The term “heteroatom-substituted Cₙ-aralkylamino” refers to a radical, having a single nitrogen atom as the point of attachment, further having at least one or two saturated carbon atoms attached to the nitrogen atom, further having a total of n carbon atoms, 0, 1, or more than one hydrogen atom, at least one additional heteroatom, that is, in addition to the nitrogen atom at the point of attachment, wherein at least one of the carbon atom incorporated into an aromatic ring, further wherein each heteroatom is independently selected from the group consisting of N, O, F, Cl, Br, I, Si, P, and S. For example, a heteroatom-substituted C₇-C₁₀-aralkylamino has 7 to 10 carbon atoms. The term “heteroatom-substituted Cₙ-aralkylamino” includes groups, having the structure —NHR, in which R is a heteroatom-substituted Cₙ-aralkyl, as that term is defined above.

The term “amido” includes straight-chain amido, branched-chain amido, cycloamido, cyclic amido, heteroatom-unsubstituted amido, heteroatom-substituted amido, heteroatom-unsubstituted Cₙ-amido, heteroatom-substituted Cₙ-amido, alkylcarbonylamino, arylcarbonylamino, alkoxycarbonylamino, aryloxycarbonylamino, acylamino, alkylaminocarbonylamino, arylaminocarbonylamino, and ureido groups. The term “heteroatom-unsubstituted Cₙ-amido” refers to a radical, having a single nitrogen atom as the point of attachment, further having a carbonyl group attached via its carbon atom to the nitrogen atom, further having a linear or branched, cyclic or acyclic structure, further having a total of n carbon atoms, 1 or more hydrogen atoms, a total of one oxygen atom, a total of one nitrogen atom, and no additional heteroatoms. For example, a heteroatom-unsubstituted C₁-C₁₀-amido has 1 to 10 carbon atoms. The term “heteroatom-unsubstituted Cₙ-amido” includes groups, having the structure —NHR, in which R is a heteroatom-unsubstituted Cₙ-acyl, as that term is defined above. The group, —NHC(O)CH₃, is a non-limiting example of a heteroatom-unsubstituted amido group. The term “heteroatom-substituted Cₙ-amido” refers to a radical, having a single nitrogen atom as the point of attachment, further having a carbonyl group attached via its carbon atom to the nitrogen atom, further having a linear or branched, cyclic or acyclic structure, further having a total of n aromatic or nonaromatic carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C₁₀-amido has 1 to 10 carbon atoms. The term “heteroatom-substituted Cₙ-amido” includes groups, having the structure —NHR, in which R is a heteroatom-unsubstituted Cₙ-acyl, as that term is defined above. The group, —NHCO₂CH₃, is a non-limiting example of a heteroatom-substituted amido group.

The term “alkylthio” includes straight-chain alkylthio, branched-chain alkylthio, cycloalkylthio, cyclic alkylthio, heteroatom-unsubstituted alkylthio, heteroatom-substituted alkylthio, heteroatom-unsubstituted Cₙ-alkylthio, and heteroatom-substituted Cₙ-alkylthio. The term “heteroatom-unsubstituted Cₙ-alkylthio” refers to a group, having the structure —SR, in which R is a heteroatom-unsubstituted Cₙ-alkyl, as that term is defined above. The group, —SCH₃, is an example of a heteroatom-unsubstituted alkylthio group. The term “heteroatom-substituted Cₙ-alkylthio” refers to a group, having the structure —SR, in which R is a heteroatom-substituted Cₙ-alkyl, as that term is defined above.

The term “alkenylthio” includes straight-chain alkenylthio, branched-chain alkenylthio, cycloalkenylthio, cyclic alkenylthio, heteroatom-unsubstituted alkenylthio, heteroatom-substituted alkenylthio, heteroatom-unsubstituted Cₙ-alkenylthio, and heteroatom-substituted Cₙ-alkenylthio. The term “heteroatom-unsubstituted Cₙ-alkenylthio” refers to a group, having the structure —SR, in which R is a heteroatom-unsubstituted Cₙ-alkenyl, as that term is defined above. The term “heteroatom-substituted Cₙ-alkenylthio” refers to a group, having the structure —SR, in which R is a heteroatom-substituted Cₙ-alkenyl, as that term is defined above.

The term “alkynylthio” includes straight-chain alkynylthio, branched-chain alkynylthio, cycloalkynylthio, cyclic alkynylthio, heteroatom-unsubstituted alkynylthio, heteroatom-substituted alkynylthio, heteroatom-unsubstituted Cₙ-alkynylthio, and heteroatom-substituted Cₙ-alkynylthio. The term “heteroatom-unsubstituted Cₙ-alkynylthio” refers to a group, having the structure —SR, in which R is a heteroatom-unsubstituted Cₙ-alkynyl, as that term is defined above. The term “heteroatom-substituted Cₙ-alkynylthio” refers to a group, having the structure —SR, in which R is a heteroatom-substituted Cₙ-alkynyl, as that term is defined above.

The term “arylthio” includes heteroatom-unsubstituted arylthio, heteroatom-substituted arylthio, heteroatom-unsubstituted Cₙ-arylthio, heteroatom-substituted Cₙ-arylthio, heteroarylthio, and heterocyclic arylthio groups. The term “heteroatom-unsubstituted Cₙ-arylthio” refers to a group, having the structure —SAr, in which Ar is a heteroatom-unsubstituted Cₙ-aryl, as that term is defined above. The group, —SC₆H₅, is an example of a heteroatom-unsubstituted arylthio group. The term “heteroatom-substituted Cₙ-arylthio” refers to a group, having the structure —SAr, in which Ar is a heteroatom-substituted Cₙ-aryl, as that term is defined above.

The term “aralkylthio” includes heteroatom-unsubstituted aralkylthio, heteroatom-substituted aralkylthio, heteroatom-unsubstituted Cₙ-aralkylthio, heteroatom-substituted Cₙ-aralkylthio, heteroaralkylthio, and heterocyclic aralkylthio groups. The term “heteroatom-unsubstituted Cₙ-aralkylthio” refers to a group, having the structure —SAr, in which Ar is a heteroatom-unsubstituted Cₙ-aralkyl, as that term is defined above. The group, —SCH₂C₆H₅, is an example of a heteroatom-unsubstituted aralkyl group. The term “heteroatom-substituted Cₙ-aralkylthio” refers to a group, having the structure —SAr, in which Ar is a heteroatom-substituted Cₙ-aralkyl, as that term is defined above.

The term “acylthio” includes straight-chain acylthio, branched-chain acylthio, cycloacylthio, cyclic acylthio, heteroatom-unsubstituted acylthio, heteroatom-substituted acylthio, heteroatom-unsubstituted Cₙ-acylthio, heteroatom-substituted Cₙ-acylthio, alkylcarbonyloxy, arylcarbonyloxy, alkoxycarbonyloxy, aryloxycarbonyloxy, and carboxylate groups. The term “heteroatom-unsubstituted Cₙ-acylthio” refers to a group, having the structure —SAc, in which Ac is a heteroatom-unsubstituted Cₙ-acyl, as that term is defined above. The group, —SCOCH₃, is an example of a heteroatom-unsubstituted acylthio group. The term “heteroatom-substituted Cₙ-acylthio” refers to a group, having the structure —SAc, in which Ac is a heteroatom-substituted Cₙ-acyl, as that term is defined above.

The term “alkylsilyl” includes straight-chain alkylsilyl, branched-chain alkylsilyl, cycloalkylsilyl, cyclic alkylsilyl, heteroatom-unsubstituted alkylsilyl, heteroatom-substituted alkylsilyl, heteroatom-unsubstituted Cₙ-alkylsilyl, and heteroatom-substituted Cₙ-alkylsilyl. The term “heteroatom-unsubstituted Cₙ-alkylsilyl” refers to a radical, having a single silicon atom as the point of attachment, further having one, two, or three saturated carbon atoms attached to the silicon atom, further having a linear or branched, cyclic or acyclic structure, containing a total of n carbon atoms, all of which are nonaromatic, 5 or more hydrogen atoms, a total of 1 silicon atom, and no additional heteroatoms. For example, a heteroatom-unsubstituted C₁-C₁₀-alkylsilyl has 1 to 10 carbon atoms. An alkylsilyl group includes dialkylamino groups. The groups, —Si(CH₃)₃ and —Si(CH₃)₂C(CH₃)₃, are non-limiting examples of heteroatom-unsubstituted alkylsilyl groups. The term “heteroatom-substituted Cₙ-alkylsilyl” refers to a radical, having a single silicon atom as the point of attachment, further having at least one, two, or three saturated carbon atoms attached to the silicon atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the silicon atom at the point of attachment, wherein each additional heteroatom is independently selected from the group consisting of N, O, F, Cl, Br, I, Si, P, and S. For example, a heteroatom-substituted C₁-C₁₀-alkylsilyl has 1 to 10 carbon atoms.

### V. Containers

In certain aspects, quartz or glass cuvettes may be used. In other aspects, single-use polystyrene (PS) and Brand UV (UV) cuvettes may be used. Although differences between controls and potency assays are smaller than those obtained with quartz cuvettes, this may be offset by using much higher potencies (50 M) than those used originally with quartz cuvettes (200).

### VI. Solvents

In some embodiments, an appropriate solvent is used to dissolve a polar and polarizable molecule. One of ordinary skill in the art can choose an appropriate solvent without undue experimentation. The solvent may be an ionic solvent or a non-ionic solvent. A UV-visible spectra of the solution containing the polar and polarizable molecule in the solvent shows a change in absorbance, such as one or more of absorptions at wavelengths that is different from the solution of the polar and polarizable molecule in a solution containing a homeopathic solution (i.e. a color change), or different intensities at the same wavelength. Exemplary non-ionic solvents include, but are not limited to, water, ethanol, and tert-butyl alcohol. Exemplary ionic solvents include, but are not limited to 1-ethyl-3-methylimidazolium acetate, 1-butyl-3-methylimidazolium hexafluorophosphate, benzyldimethyltetradecylammonium chloride, tetrabutylphosphonium methanesulfonate, 1-butyl-4-methylpyridinium hexafluorophosphate, 1-butyl-1-methylpyrrolidinium bromide, and triethylsulfonium bis(trifluoromethylsulfonyl)imide. In some aspects, the water may be distilled water, deionized water, or reverse osmosis water (ROW).

In certain aspects, water or non-aqueous solvents may be used in the methods described herein. In other aspects, it is contemplated that any organic solvents may be used.

### VII. Detection Methods

Disclosed herein is a simple, optical sensor containing a polar and polarizable molecule for characterization of a homeopathic sample. The sensing mechanism of the sensor relies on the physicodynamic properties of the compound, which undergoes a color or fluorescence change in the presence of a homeopathic solution, as compared with a control without the homeopathic solution.

The disclosed method of detecting a homeopathic solution in a sample includes contacting the sample with a polar and polarizable molecule, wherein the polar and polarizable molecule undergoes a shift, continuous or in particular aspects, discontinuous, in spectral absorbance when in contact with the homeopathic solution in the sample as compared to the absorbance of the polar and polarizable molecule in a control solution without the addition of a homeopathic solution. The spectral absorbance and/or emission may be detected. A change in the spectral absorbance, for example relative to a control, indicates the presence of a homeopathic solution in the sample.

Thus, disclosed is a method of detecting a homeopathic solution in a sample. The method involves contacting the sample with a polar and polarizable molecule that undergoes a shift in spectral absorbance when in contact with the homeopathic solution in the sample.

Upon contact with the sample, optical responses, or spectral absorbance, can be detected, which in some examples are recorded. The optical response generated can be intensity changes, spectral shift, and time-dependent variations associated with the sensor elements upon exposure, for example to a sample or a reference fluid (such as methanol, ethanol, DMF, DCM, acetone, toluene, etc., as well as buffered aqueous solutions). In some examples, a light source, for example multicrhomatic light source is used as an excitation source for the polar and polarizable molecule. The light can be filtered by an excitation filter before reaching the sample. Spectral absorbance can be detected by any methods known in the art, for example using a spectrophotometer.

In some embodiments, the spectral absorbance is compared to a control. In some embodiments, controls for use in the disclosed methods include one or more values indicative of a known concentration of a homeopathic solution in a control sample, for example as a calibration curve. In some embodiments, controls for use in the disclosed methods include one or more values indicative of a known concentration of a homeopathic solution in a control sample, for example as a calibration curve. In some examples the control is one of more control samples with a known concentration of a homeopathic solution or values derived therefrom, for example as a calibration curve. In some embodiments, the control is a calibration curve.

In certain embodiments, the method comprises an optical absorption technique. Additionally or alternatively, the method comprises a fluorescent technique using a compound such as Nile Red or Brooker's merocyanine. In certain examples, the detection can include detection with a spectrophotometer in the ultraviolet visible wavelength range. In some examples, the detected spectral absorbance is used for quantification of a homeopathic solution in the sample. In some embodiments, the method further includes quantifying the amount of a homeopathic solution in the sample. A sensor may be used for the detection and quantification of a homeopathic solution in a sample.

Detection by one or more of the plurality of detection methods can provide an image of the substrate or a representation thereof. The image can be a photograph or digital image. A representation thereof can include a false-color image where features such as texture, depth, surface roughness, luminescence intensity, and the like, are represented by colors or other indicia.

A system for carrying out the methods disclosed herein can include an input element for receiving one or more images of a first detectable array or a representation thereof. The input element can be any element that is used to both create and receive an image or representation thereof, such as a photo-scanner, or an element that is used solely to receive a digital representation of an image, such as a computer, web browser, cloud-computing environment, and the like.

The system can also include a database comprising one or more images of a plurality of second detectable arrays or representations thereof. The database can be stored physically, such as a photograph album containing images, but is more commonly stored digitally. For example the database can be stored on one or more computers, computer servers, computer-readable storage devices, such as hard drives, USB drives, and the like, or in a cloud-computing environment.

The system can further include a comparison element for comparing the one or more images of the first detectable array or representation thereof to the one or more images of the plurality of second detectable arrays or representations thereof. The comparison element can be a simple physical element, such as one or more photograph albums comprising one or more images of a plurality of second detectable arrays or representations thereof for facilitating a visual comparison of the image of the first detectable array or representation thereof with the plurality of second detectable arrays or representations thereof.

In some embodiments, the comparison element may include software that performs a series of steps to recognize features, such as color, size, location, and the like, and patterns of such features, of the image of the first detectable array or representation thereof, and compares those features to the one or more images of the plurality of second detectable arrays or representations thereof. The software can be executed on any suitable device, including a computer, mobile computer, mobile or stationary telephone equipped with software-executing capabilities (e.g., “smart-phone”), tablet computer, wearable computer, and the like. The software can also be executed in whole or in part from a computer server or a cloud-computing environment, which need not be in the same location as the input element. Thus, the comparison element can compare the one or more images of the a homeopathic solution or representation thereof to the one or more images of the control solution or representations thereof by, for example, executing local software to perform this comparison or executing or causing to be executed software that is located in another location to perform this comparison.

In some cases, the comparison element and the database can be the same device, although this is not required unless otherwise specified. Also, the comparison element need not comprise software; the comparison element can also be, for example, a photograph album or physical representation that facilitates comparison of the one or more images of the sample solution or representation thereof to the one or more images of the control solution or representations thereof.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

To date, nine different polar and polarizable molecules have been investigated, four positively solvatochromic, three negatively solvatochromic, and one halochromic.

Potencies of glycerol were chosen as the primary example of homeopathic remedy for assay. Glycerol is a low molecular weight compound that can be obtained at very high purity levels, is pharmacologically inactive in material doses and is fully miscible with both water and ethanol.

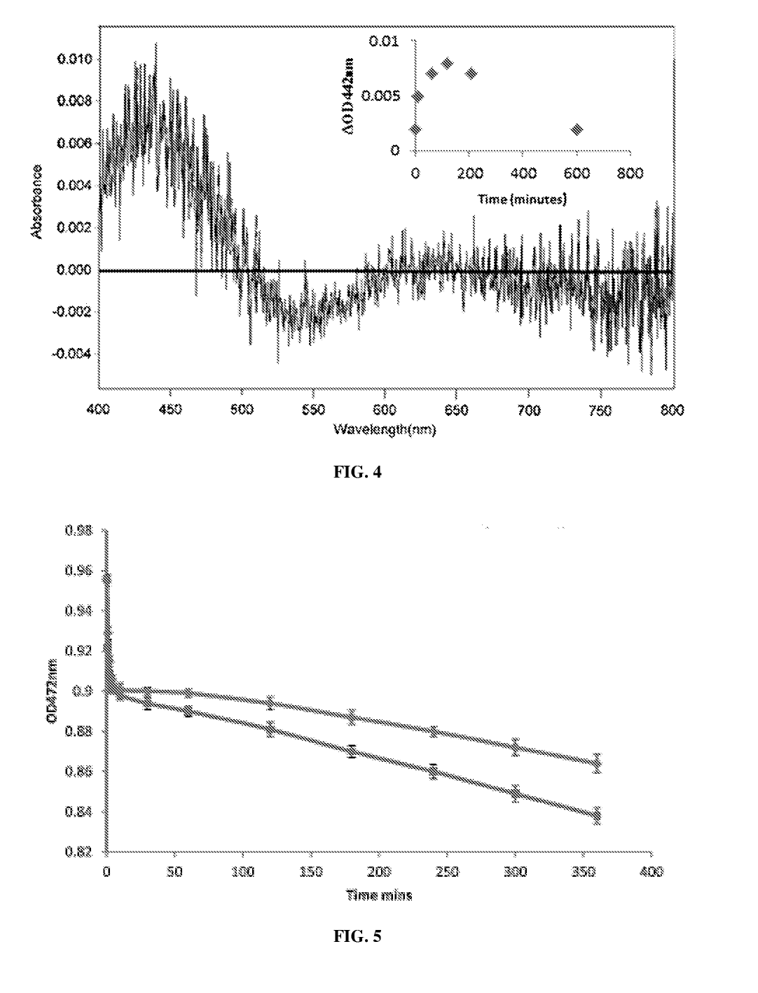

Results with dye ET33 FIG. 4 shows a difference spectrum obtained on adding an aliquot of glycerol 50 M (sample cuvette) and an equivalent volume of control (reference cuvette), to solutions of the negatively solvatochromic dye ET33 dissolved in ethanol. Scans were then repeatedly made from 400 nm to 800 nm over several hours (see Materials and methods). The scan shown was taken at 120 min after mixing. There is a relative decrease at c.542 nm and a relative increase at c.442 nm which builds up over time.

The difference is minimal at t=0 but slowly reaches a maximum at 120 min (see FIG. 4 insert). This difference spectrum should be considered in relation to the spectrum of ET33 in ethanol which has a broad peak at 472 nm. ET33 aggregates in solution to produce stepped aggregates (J-aggregates) that absorb at longer wavelengths than monomer (bathochromically shifted).20 Solutions of ET33 are therefore an equilibrium mixture of monomer and aggregates, hence the broad nature of the absorption spectrum of ET33. What FIG. 4 appears to be showing therefore is that potency is causing ET33 to disaggregate to produce more monomer.

Support for this proposition comes from assays utilising divalent cations. Strontium ions interact with the phenoxide moiety of ET3325 (FIG. 1) resulting in a loss of absorbance. This loss occurs in two stages: a rapid first phase due to the direct interaction of strontium ions with free dye, followed by a second phase arising from rate-limiting disaggregation of dye. If potency causes a greater level of disaggregation of dye then one might expect more rapid loss of absorbance in the presence of potency. This is indeed what is seen. FIG. 5 shows assays in which either control plus strontium ions or glycerol 50M plus strontium ions are added to solutions of ET33 in ethanol. Loss of absorbance is more rapid in the presence of potency, supporting the idea that potency enhances disaggregation of dye.

Difference spectra of ET33 in tert-butyl alcohol with and without potency show a decrease at c.615 nm and an increase at c.490 nm (the absorbance peak of ET33 in tert-butyl alcohol is at 548 nm). As with assays in ethanol, glycerol 50 M appears to be causing enhanced disaggregation of dye. What these results in ethanol and tert-butyl alcohol show is that bulk water is not essential to manifest a potency effect. More specifically, as tert-butyl alcohol very poorly hydrogen bonds, it is likely that hydrogen bonding is not essential in order to manifest a potency effect.

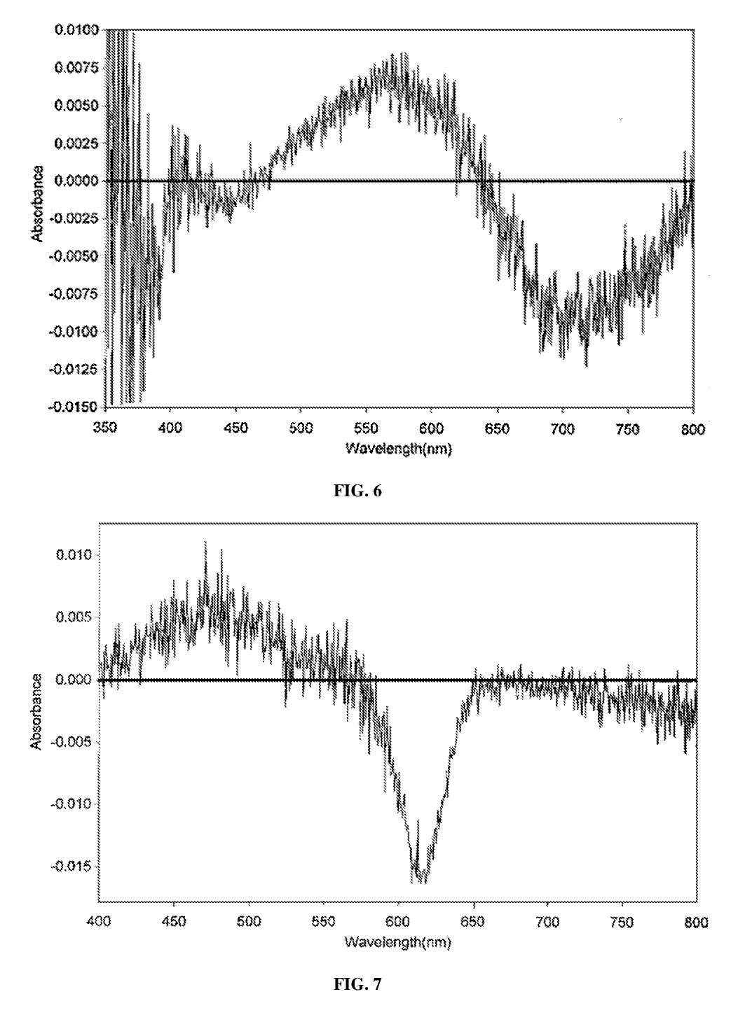

Results with dye ET30. Difference spectra of ET30 (FIG. 1) in water, ethanol and tert-butyl alcohol indicate enhanced disaggregation in the presence of glycerol 50 Min all three cases. The difference spectrum in tert-butyl alcohol is one of the largest seen in any of the dye/solvent combinations examined (FIG. 6). As with ET33, difference spectra tend to develop over time, although on some occasions evidence of a difference spectrum is present even from t=0. Scans in most of the dye/solvent combinations tested tend to show a return to base line with loss of any difference spectrum overnight, indicating that the potency effect is one that slowly builds up over time, reaching a maximum somewhere between 1 and 2 h and then slowly disappears overnight.

Consistent with the results seen with ET33, glycerol 50 M also increases the rate of complexation of strontium ions with ET30, through enhanced disaggregation.

Results with dye BDN. Complementary evidence to that with ET33 and ET30 is seen with the positively solvatochromic dye BDN. FIG. 7 shows a difference spectrum of BDN in ethanol with and without glycerol 50 M. Conditions are exactly the same as in FIG. 4 except BDN has been substituted for ET33. Scans were performed at the same time intervals as for ET33. The scan shown was performed at t=30 min after mixing. There is a relative decrease at c.615 nm and a relative increase at c.480 nm. This difference spectrum should be seen in relation to the absorption spectrum of BDN in ethanol which shows two poorly differentiated peaks at 484 nm and 564 nm corresponding to aggregate and monomer peaks respectively. BDN in solution produces aggregates (H-aggregates) that absorb at shorter wavelengths than monomer. As with ET33, solutions of BDN are equilibrium mixtures of monomer and aggregates. However, in distinction to ET33 the aggregates in BDN are hypsochromically shifted. The difference spectrum in FIG. 7 therefore appears to show that glycerol 50 M is promoting dye aggregation in BDN.

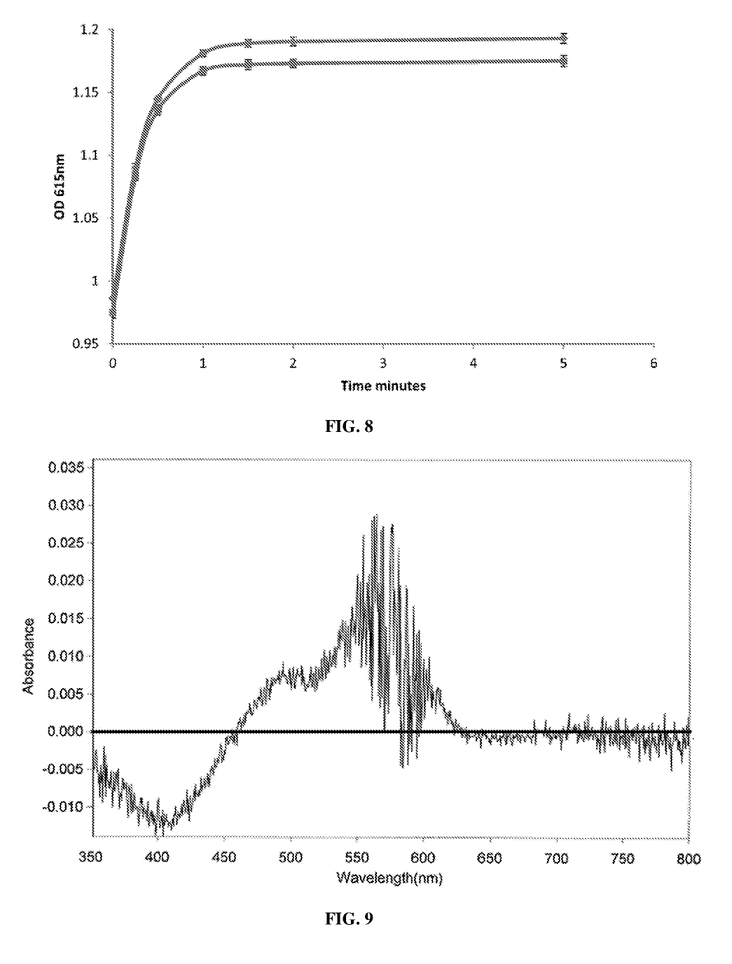

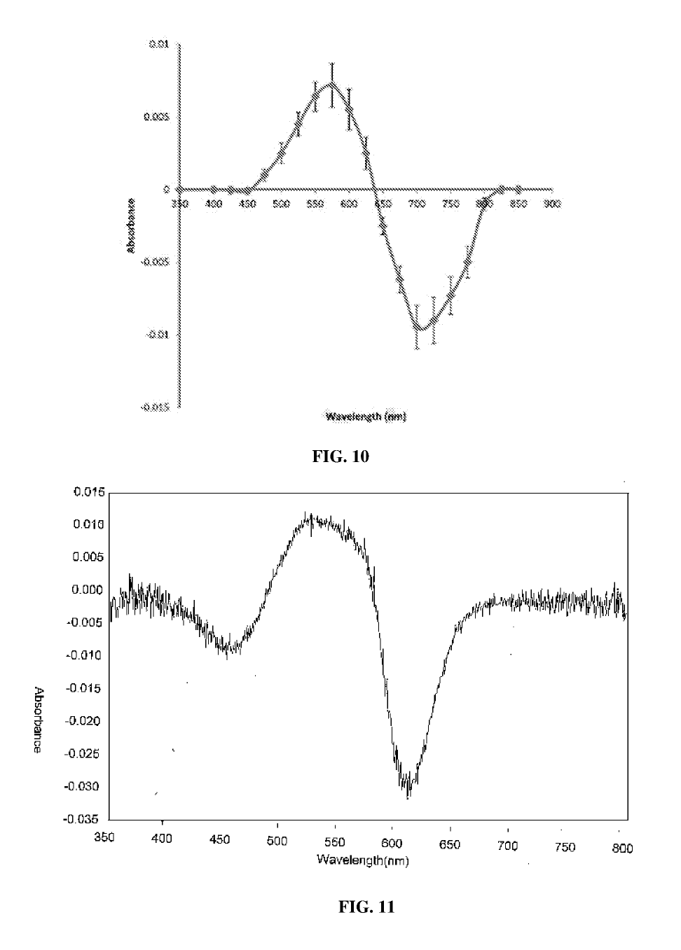

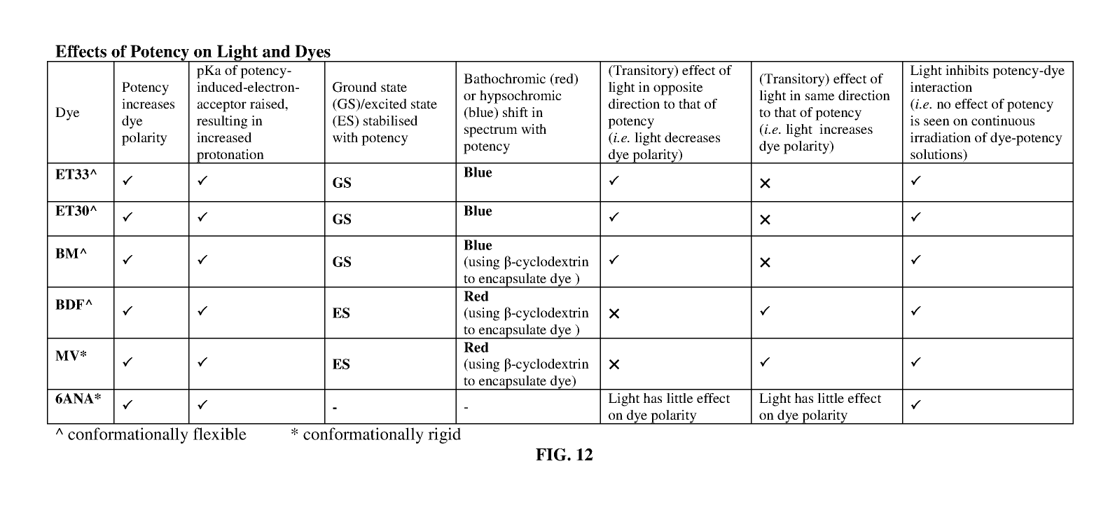

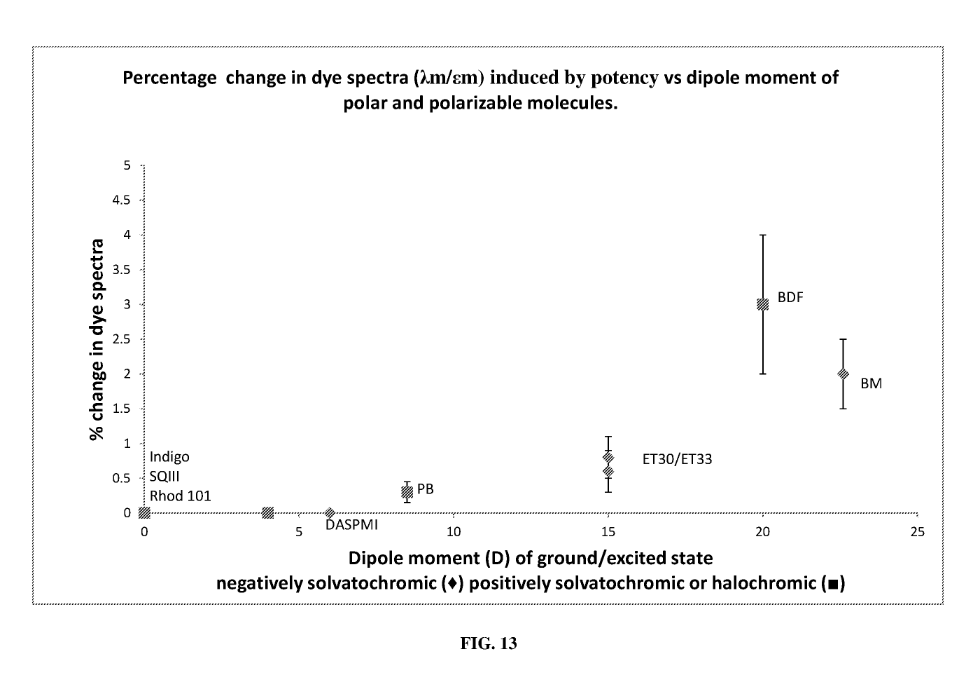

Divalent cations interact with BDN to produce a rise in absorbance at 615 nm and a decrease in absorbance at 484 nm. As with ET33 and ET30, this complexation has a second slower phase arising from rate-limiting disaggregation of dye. If potency is enhancing dye aggregation then one would expect potency to reduce the rate of increase in absorbance at 615 nm compared with that in the absence of potency. This is indeed what is seen. FIG. 8 shows assays in which either control plus strontium ions or glycerol 50M plus strontium ions are added to solutions of BDN in ethanol. Gain of absorbance is slower in the presence of potency, supporting the idea that potency protects BDN from complexation with strontium ions through enhanced dye aggregation.

Difference spectra of BDN in water, ethanol and tert-butyl alcohol with glycerol 50 M all show increases at shorter wavelengths and decreases at longer wavelengths corresponding to enhanced dye aggregation, irrespective of solvent. Solvent does not therefore appear to be playing a direct part in the action of glycerol 50 M. Furthermore, if potency were acting on either ET33 or BDN through changes in solvent polarity or hydrogen bonding capabilities the dyes should not be affected in opposite ways. Both dyes tend to aggregate more strongly as solvent polarity decreases and disaggregate as solvent hydrogen bonding capability increases.

Results with other solvatochromic dyes—Further evidence of interactions between glycerol 50 M and solvatochromic dyes comes from experiments using the negatively solvatochromic dye BM and the positively solvatochromic dyes NR and PB (FIG. 1). All these dyes produce significant difference spectra in the presence of potency. BM is particularly interesting as in water, ethanol and tert-butyl alcohol, glycerol 50 M promotes aggregation. This is in contrast to that seen with ET30 and ET33. Aggregates are bathochromically shifted with respect to monomer in BM, so difference spectra show increases at longer wavelengths and decreases at shorter wavelengths. FIG. 9 shows the difference spectrum in tert-butyl alcohol.

The difference spectra of NR and PB with glycerol 50M in water, ethanol and tert-butyl alcohol indicate potency promotes disaggregation of both dyes in water, ethanol and tert-butyl alcohol. Table 1 lists the effects of glycerol 50M on the spectra of all the solvatochromic dyes examined in this study, giving the positions of maximum absorbance changes on addition of potency. It should be noted that for dyes ET30, ET33 and BM in water only single difference maxima are given. This is because all dyes absorb strongly and non-specifically below c. 400 nm, rendering accurate difference spectra beyond the capabilities of the instrument below this wavelength.

TABLE 1
Table showing the positions of maximum absorbance changes (difference spectra) on addition of
glycerol 50M to solvatochromic dyes used in this study in three different solvent systems (water, ethanol and
tert-butyl alcohol). Absorbance maxima for dyes in respective solvents are given in plain type whilst the
positions of maximum absorbance change on addition of potency are given in italics.

| | Solvent system | | |
|---|---|---|---|
| Dye | Water | Ethanol | Tert-butyl alcohol |

Negatively solvatochromic

| | | | |
|---|---|---|---|
| ET33 | Absorbance maxima 409 nm (broad) | Absorbance maxima 472 nm (broad) | Absorbance maxima 548 nm (broad) |
| | Decrease at c.380-420 nm with potency | Increase at 442 nm and decrease | Increase at 490 nm and decrease |
| | | at 542 nm with potency | at 615 nm with potency |
| ET30 | Absorbance maxima 450 nm (broad) | Absorbance maxima 550 nm (broad) | Absorbance maxima 650 nm (broad) |
| | Decrease at c.450 nm with potency | Increase at 450 nm and decrease | Increase at 580 nm and decrease at |
| | | at 580 nm with potency | 715 nm with potency |
| BM | Absorbance maxima | Absorbance maxima | Absorbance maxima |
| | 380 nm (monomer) | 400 nm (monomer) | 402 nm (monomer) |
| | 442 nm (aggregate) | 513 (aggregate) | c.496 nm (minor peak) (aggregate) |
| | Increase at 450 nm with potency | Decrease at 390 nm and increase | 576 nm (aggregate) |
| | | at 510 nm with potency | Decrease at 404 nm and increase |
| | | | at 500 nm and 570 nm with potency |

Positively solvatochromic

| | | | |
|---|---|---|---|
| BDN | Absorbance maxima | Absorbance maxima | Absorbance maxima |
| | 490 nm (aggregate) | 484 nm (aggregate) | 459 nm (aggregate) |
| | 614 nm (monomer) | 564 nm (overlapping aggregate | 537 nm (aggregate) |
| | Increase at 460 nm and | and monomer peaks?) | 609 nm (monomer) |
| | decrease at 620 nm with potency | Increase at 480 nm and decrease | Increase at 460 nm and 535 nm and |
| | | at 615 nm with potency | decrease at 620 nm with potency |
| NR | Absorbance maxima | Absorbance maxima 550 nm (broad) | Absorbance maxima 538 nm (broad) |
| | 530 nm (aggregate) | Decrease at c.475 nm and increase | Decrease at c.465 nm and increase |
| | 593 nm (monomer) | at c.560-570 nm with potency | at c.560 nm with potency |
| | Decrease at c.480 nm and increase | | |
| | at c.560 nm with potency | | |
| PB | Absorbance maxima 658 nm (broad) | Absorbance maxima | Absorbance maxima 601 nm (broad) |
| | Decrease at c.520 nm and increase | 608 nm (broad) | Decrease at c.520 nm increase at |
| | at c.670 nm with potency | Decrease at c.540 nm and increase | c.620-660 nm with potency |
| | | at c.680 nm with potency | |

Table 2, in turn, gives a summary, derived from difference spectra, of the deduced supramolecular effects of glycerol 50 M on dyes ET30, ET33, BM, BDN, NR, PB tested in water, ethanol and tert-butyl alcohol.

TABLE 2
Table showing the deduced effect of glycerol 50M on the
supramolecular chemistry (enhanced aggregation or enhanced
disaggregation) of all solvatochromic dyes used in this study

Effect of glycerol 50M potency

| | | | |
|---|---|---|---|
| Dye | Water | Ethanol | Tert-butyl alcohol |

Negatively solvatochromic

| | | | |
|---|---|---|---|
| ET33 | Disaggregation | Disaggregation | Disaggregation |
| ET30 | Disaggregation | Disaggregation | Disaggregation |
| BM | Aggregation | Aggregation | Aggregation |

Positively solvatochromic

| | | | |
|---|---|---|---|
| BDN | Aggregation | Aggregation | Aggregation |
| NR | Disaggregation | Disaggregation | Disaggregation |
| PB | Disaggregation | Disaggregation | Disaggregation |

6ANA (halochromic, and not solvatochromic) and MV exhibit significant responses to homeopathic potencies. Both of these compounds have substantial dipole moments. MV is >18D in its ground state and >22D in its excited state. Evidence indicates that the degree of response to homeopathic potencies correlates with dipole moment of the polar and polarizable molecule. Molecules with larger dipole moments may facilitate analysis of homeopathic potencies.

Materials and Methods

Materials—Polar and polarizable molecules 2,6-Dichloro-4-(2,4,6-triphenylpyridinium-1-yl)-phenolate (ET33), 2,6-Diphenyl-4-(2,4,6-triphenylpyridinium-1-yl)-phenolate (ET30), N,Ndimethylindoaniline/Phenol Blue (PB), 9-diethylamino-5H-benzo[a]phenoxazime-5-one/Nile Red (NR) and 4-(Bis-(4-(dimethylamino)phenyl)methylene)-1(4H)-naphthalenone (BDN), all solvatochromic, were obtained from Sigma Aldrich UK and used as provided. 4-[(E)-2-(1-methylpyridinium-4-yl)ethenyl]phenolate/Brooker's merocyanine (BM, solvatochromic) was synthesised and provided by Innovapharm Ltd., Kiev, Ukraine and its structure and purity confirmed by NMR. BDF (solvatochromic) was a gift from Dr. Ana Costero, Institutio Interuniversitario de Reconocimiento Molecular y Desarrollo Tecnologico, Spain. Nonsolvatochromic dyes Patent Blue VF, Green S, Cresol Red and Sulforhodamine 101 were obtained from Sigma Aldrich or Fisher Scientific, UK. Strontium chloride, citric acid, sodium phosphate and sodium borate, used to make buffer solutions between pH 4 and 10, as well as ethanol and tert-butyl alcohol were obtained from Sigma Aldrich, UK unless specified otherwise and were of the highest purity available.

Reverse osmosis water (ROW) was used throughout this study and had a resistivity of 15 MΩ cm (checked daily).

Disposable high purity optical PS cuvettes 1.5 ml and 4.5 ml capacity/10 mm pathlength with polyethylene (PE) air-tight stoppers were obtained from Elkay Laboratory Products, UK. Disposable high purity UV 1.5 ml and 4.5 ml capacity/10 mm pathlength cuvettes (UV) with PE air-tight stoppers were obtained from Brand GMBH, Germany. High purity/low leachable trace element Nalgene PE, and fluorinated ethylenepropylene (FEP) bottles were obtained from Fisher Scientific, UK.

Solution storage—All solutions were made up and stored in FEP or PE bottles. Ethanol and tert-butyl alcohol were used from the bottles in which they were provided or transferred to PE bottles. Concentrated dye stocks in dimethylsulfoxide (DMSO) or dye solutions in ROW, ethanol or tert-butyl alcohol were stored in FEP or PE bottles at room temperature. Working dye solutions were made by dissolving dye directly into solvent (ROW, ethanol or tert-butyl alcohol) or by adding an aliquot of concentrated dye stock in DMSO to solvent. In both cases dye solutions were left to equilibrate overnight before use. Concentrations of dyes used for difference spectra and assays with strontium ions were as follows: ET33—245 μM in ROW, ethanol and tert-butyl alcohol; ET30—245 μM in ethanol and tert-butyl alcohol. ET30 is poorly soluble in ROW. Solutions were made up in 20 mM borate buffer pH 10 and centrifuged to remove any precipitate; BDN—80 μM in ROW, ethanol and tert-butyl alcohol. BDN slowly precipitates in ROW, so solutions were made up and used within an hour; BM—125 μM in ethanol, 245 μM in ROW and 125 μM in tert-butyl alcohol; NR—16 μM in ROW, ethanol and tert-butyl alcohol; PB—62 μM in ROW, ethanol and tert-butyl alcohol. Dye concentrations were chosen so as to give absorbances of c.1.0 at their absorbance maxima. At this absorbance level the Unicam UV-500 spectrophotometer used in this study can comfortably handle difference spectra. No light or temperature induced degradation (determined by daily monitoring of visible spectra) of ET33, ET30, PB, BDN, BM or NR was observed over the period of this study. As a precaution solutions of BM and NR were kept in the dark as both dyes are fluorescent and potentially subject to light-induced degradation.

Homeopathic potencies—Serially diluted and succussed solutions (homeopathic potencies) of a range of compounds, including glycerol 50 M, were obtained from Helios Homeopathy Ltd Tunbridge Wells, UK or Ainsworths Homeopathic Pharmacy, London, UK and were diluted 10 fold into 90% ethanol/10% ROW to ensure consistent solvent composition. Potencies are sold as made in 90% ethanol/10% ROW, but the above step was taken as a further precaution to ensure solvent equivalence with control solutions. Glycerol 50 M is a homeopathic potency prepared by the Korsakoff method. Glycerol is serially diluted and succussed by hand up to the 200c potency. Thereafter potentisation is performed mechanically. At each step a portion of solution is first diluted 100 fold, and then subjected to 10 succussion strokes. 50 M means the homeopathic medicine has gone through 50,000 such cycles. A total of 500,000 succussion strokes have therefore been imparted with an effective dilution factor of 100⁵⁰⁰⁰⁰. ROW is used throughout the potentisation process.

Control solutions—Controls consisted of 90% ethanol/10% ROW alone. Both potencies and controls were kept in the same 3.5 ml amber molded glass bottles used by Helios Homeopathy Ltd (obtained from the Homeopathic Supply Company Ltd, code MPB) under the same conditions at room temperature. Any impurities leaching out of the glassware should therefore be present at the same levels in both control and potency solutions. Independent analysis by ICP-OES for levels of Aluminum, Boron, Calcium, Iron, Potassium, Magnesium, Sodium, Silicon and Titanium by LGC Health Sciences, UK confirmed the same level of each element in both control and potency bottles (kept at room temperature for 6 months prior to analysis). Furthermore, at the levels of elements detected (all <2 μg/ml of potency and control solutions) no effect on either dye difference spectra or assays involving dyes and strontium ions (see Experimental procedures below) was observed in an independent study in which salts of the above elements were deliberately added to assay solutions. Levels of other elements in control and potency solutions were below the detection limits of the ICP-OES instrumentation.

Instrumentation—Assays and spectra were recorded on a Unicam UV500 uv/vis double-beam spectrophotometer run with preloaded Visionlite software capable of analysing curves and providing data points at set time intervals as they are generated. Manufacturer's specifications state an accuracy of ±0.001 at an absorbance of 1.0, with a wavelength accuracy of 0.5 nm and resolution of 2 nm.

Experimental procedures—Assays were performed in single-use 1.5 ml capacity PS cuvettes (assays in ROW and ethanol) or UV cuvettes (assays in ethanol and tert-butyl alcohol) as described above. Quartz and glass cuvettes have also been used and similar results obtained. Tests showed no evaporation of contents occurred over the time periods of assays from either cuvette type using the stoppers described.

Difference spectra of dyes were typically performed as follows. 2.95 ml of dye solution at concentrations giving an absorbance of c.1.0 (see Solution storage above) was dispensed into sample and reference cuvettes and the spectrophotometer set to zero across the range of wavelengths to be assessed (generally 350 or 400 nm to 700 or 800 nm depending upon the dye being investigated). 50 μl of control solution (90% ethanol/10% ROW) was then added to the reference cuvette and 50 μl of potency solution (90% ethanol/10% ROW) added to the sample cuvette. Solutions in both cuvettes are therefore materially identical. Solutions were scanned at time intervals up to 3 h to give difference spectra of dye with potency minus dye with control. Scans of solutions left overnight were also performed.

‘Difference’ spectra in which 50 μl of control solution was added to both cuvettes demonstrated no effect over comparable time scales compared with dye solutions to which potency had been added.

Assays with strontium ions were carried out as follows. To 2.95 ml of dye solution in PS or UV cuvettes was added 50 μl of either control solution or potency solution together with, in both cases, 1 μl of a 210 mM solution of strontium chloride. Absorbance was then followed at a set wavelength to monitor changes in the interaction of dye with strontium ions. The spectrophotometer was zeroed with solvent alone in the respective cuvette type prior to assay. All presented data show average values of at least 20 assays under any one set of conditions.

PS or UV cuvettes have been used throughout this study. Primarily this is because they are single-use and therefore remove any possibility for cross-contamination by residual potency potentially encountered with reusable quartz cuvettes. Laboratory temperature was maintained at 21±1° C. for assays involving ROW and ethanol and 24±1° C. for assays involving tert-butyl alcohol, using a combination of background heating and air conditioning.

The highest purity ROW, ethanol and tert-butyl alcohol were used in all assays. It is perhaps worth noting however that on addition of 50 μl of either control or potency solutions to 2.95 ml of dye solution the relative levels of host solvent drop slightly because of the introduction of 45 μl of ethanol and 5 μl of ROW. Bulk solvent is therefore at a level of 98.5% in the case of those assays performed in ROW, 99.8% in the case of those assays performed in ethanol and 98.33% in the case of those assays performed in tert-butyl alcohol. As these small changes occur in both control and sample solutions they have not been taken into account, especially as potency effects appear to be solvent-independent

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Aabel S, Fossheim S, Rise F. Nuclear magnetic resonance (NMR) studies of homeopathic solutions. Br Homeopath J 2001; 90: 14-20.
Assumpcao R. Electrical impedance and HV plasma images of high dilutions of sodium chloride. Homeopathy 2008; 97: 129-133.
Balzani V, Ceroni P, Juris A. Photochemistry and photophysics: concepts, research, applications. Germany:Wiley-VCHWeinheim, 2014.
Banfield P, Hutchings M G. Chromic phenomena: technological applications of colour chemistry. 2nd edn. Cambridge, UK: RSC Publishing, 2010.
Bell I R. The evolution of homeopathic theory-driven research and metholodological toolbox. Am Homeopath 2008; 14: 56.
Bellavite P. Complexity science and homeopathy: a synthetic overview. Homeopathy 2003; 92(4): 203-212.
Belon P, Elia V, Elia L, Montanino M, Napoli E, Niccoli M. Conductometric and calorimetric studies of the serially diluted and agitated solutions. On the combined anomalous effect of time and volume parameters. J Therm Anal Calorim 2008; 93(2):459-469.
Chaplin M. The memory of water: an overview. Homeopathy 2007; 96: 143-150.
Davenas E, Beauvais F, Amara J, et al. Human basophil degranulation triggered by very dilute antiserum against IgE. Nature 1988; 333(6176): 816-818.
Eisfeld A, Briggs J S. The J- and H-bands of organic dye aggregates. Chem Phys 2006; 324: 376-384.
Elia V, Baiano S, Duro I, Napoli E, Niccoli M, Nonatelli L. Permanent physico-chemical properties of extremely diluted aqueous solutions of homeopathic medicines. Homeopathy 2004; 93: 144-150.
Hahnemann S. Organon of medicine. 5th and 6th edns. http://www.helios.co.uk/remedies/remedy-preparation, 2015.
Homeopathic Pharmacopoeia of the United States; Scholar's Choice; 2015.
Marchettini N, Del Giudice E, Voeikov V, Tiezzi E. Water: a medium where dissipative structures are produced by a coherent dynamics. J Theor Biol 2010; 265: 511-516.
Milgrom L R. Entanglement, knowledge, and their possible effects on the outcomes of blinded trials of homeopathic provings. J Altern Complement Med 2006; 12(3): 271-279.
Mishra A, Behera R K, Behera P K, Mishra B M, Behera G B. Cyanines during the 1990's. Chem Rev 2000; 100(6): 1973-2012.
Montagnier L, Aissa J, Ferris S, Montagnier J-L, Lavallee C. Electromagnetic signals are produced by aqueous nanostructures derived from bacterial DNA sequences. Interdiscip Sci Comput Life Sci 2009; 1: 81-90.
Reichardt C, Welton T. Solvent effects on the absorption spectra of organic compounds. In: Solvents and Solvent Effects in Organic Chemistry. 4th edn. Weinheim.Wiley-VCH, 2011, pp 359-424.
Reichardt C. Pyridinium N-phenolate betaine dyes as empirical indicators of solvent polarity: some new findings. Pure Appl Chem 2008; 80(7): 1415-1432.
Reichardt C. Solvatochromic dyes as solvent polarity indicators. Chem Rev 1994; 94: 2319-2358.
Reichardt C. Solvatochromism, thermochromism, piezochromism, halochromism and chiro-solvatochromism of pyridinium N-phenoxide betaine dyes. Chem Soc Rev 1992; 21: 147-153.
Rey L. Thermoluminescence of ultra-high dilutions of lithium chloride and sodium chloride. Phys A Stat Mech Appl 2003; 323: 67-74.
Speight P. Homoeopathy: a home prescriber. UK: C W Daniel Co Ltd, 1992.
Torres J L, Ruiz M A G. Stochastic resonance and the homeopathic effect. Br Homoeopath J 1996; 85: 134-140.
Wolf U, Wolf M, Heusser P, Thurneysen A, Baumgartner S. Homeopathic preparations of quartz, sulphur and copper sulphate assessed by uv-spectroscopy. Evid Based Complement Altem Med 2011; 644-654.
Wurthner F, Yao S, Debaerdemaeker T, Wortmann R. Dimerization of merocyanine dyes. Structural and energetic characterization of dipolar dye aggregates and implications for nonlinear optical materials. J Am Chem Soc 2002; 124: 9431-9447.
Yin Lo S, Geng X, Gann D. Evidence for the existence of stablewater-clusters at room temperature and normal pressure. Phys Letts A 2009; 373(42): 3872-3876.

What is claimed is:

1. A method of quantifying a homeopathic potency effect, comprising the steps of:
a) obtaining a sample solution by adding a homeopathic potency solution to a dye solution comprising a polar and polarizable molecule in a solvent;
b) obtaining a reference solution comprising an equivalent amount of the dye solution of step a);
c) obtaining a spectrum of the sample solution;
d) obtaining a spectrum of the reference solution;
e) obtaining a difference spectrum, wherein the difference spectrum is the difference between the sample solution spectrum and the reference solution spectrum; and
f) quantifying the potency effect of the homeopathic potency solution by identifying a maximum value of the difference spectrum.

2. The method of claim 1, wherein the homeopathic potency solution has been prepared by a serial dilution of between 100⁶ to 100¹⁰⁰⁰⁰⁰.

3. The method of claim 2, wherein the homeopathic potency solution has been prepared by a serial dilution of more than 100²⁰⁰.

4. The method of claim 2, wherein the homeopathic potency solution has been prepared by a serial dilution of up to 100^{50,000}.

5. The method of any of claims 1-4, wherein the polar and polarizabale molecule is a negatively solvatochromic dye.

6. The method of any of claims 1-4, wherein the polar and polarizabale molecule is a positively solvatochromic dye.

7. The method of claim 5, wherein the negatively solvatochromic dye is ET33, ET30, or BM.

8. The method of claim 6, wherein the positively solvatochromic dye is BDN, BDF, NR, or PB.

9. The method of any of claims 1-4, wherein the polar and polarizable molecule is a halochromic dye.

10. The method of claim 8.1, wherein the halochromic dye is 6-amino-2-naphthoic acid (6-ANA) or 6-amino-2-anthracenic acid (6-AAA).

11. The method of any of claims 1-8, wherein the solvent is an ionic solvent or a non-ionic solvent.

12. The method of any of claims 1-9, wherein the dye solution has a polar and polarizable molecule concentration of from 10 μM to 250 μM.

13. The method of any of claims 1-10, wherein the dye solution has a polar and polarizable molecule concentration that gives absorbance of c. 1.0 at its absorbance maxima.

14. The method of any of claims 1-11, wherein the reference solution spectrum is a control.

15. The method of any of claims 1-12, wherein the measuring step comprises obtaining a plurality of sample solution and reference solution spectra at time intervals for up to 20 days.

16. The method of any of claims 1-12, wherein the measuring step comprises obtaining a plurality of sample solution and reference solution spectra at time intervals for up to 24 hours.

17. The method of any of claims 1-14, wherein the time interval may range from one minute to one hour.

18. The method of any of claims 1-15, wherein the step of obtaining a spectrum comprises measuring an absorbance spectrum.

19. The method of any of claims 1-16, wherein the step of obtaining a spectrum comprises measuring a fluorescence spectrum.

20. The method of any of claims 1-17, wherein the sample solution has a volume of about 300 μL to 3 mL.

21. The method of any of claims 1-16, wherein the measuring comprises the use of a spectrophotometer.

22. The method of any of claims 1-19, wherein the sample solution is kept in a quartz or glass cuvette during measuring.

23. The method of any of claims 1-20, wherein the maximum value of the difference spectrum comprises the maximum of all difference spectra obtained over all time interval measurements.

24. The method of any of claims 1-21, wherein a plot of the maximum of each difference spectrum vs. time is employed to determine a maximum rate of change of difference spectra.

25. The method of any of claims 1-6 or 9-22, wherein the polar and polarizable molecule is a molecule having conjugated π-electron system through which negative charge may be delocalized.

26. The method of any of claims 1-6 or 9-22, wherein the polar and polarizable molecule is a molecule having electron donor and acceptor moieties linked through an electron delocalized system in which the compound's dipole moment in the electronic ground state is considerably different from that in the excited state.

27. The method of any of claims 1-6 or 9-22, wherein the polar and polarizable molecule is a dye selected from the group consisting of ET30, ET33, BM, BDF, BDN, PB, NR, DCM, PRODAN, DCVJ, phenol blue, a stilbazolium dye, a coumarin dye, a ketocyanine dye, analogs of Reichardt's dye, merocyanine dyes, or NDMNA.

28. The method of any of claims 1-23, wherein the solvent further comprises a buffer.

29. The method of claim 24, wherein the buffer is sodium borate/boric acid buffer, disodium citrate/trisodium citrate buffer, monopotassium phosphate/dipotassium phosphate buffer, Dimethylarsinic acid (cacodylic acid) buffer, veronal-acetate buffer, s-Collidine buffer, 3-[N-Tris(hydroxymethyl)methylamino]-2-hydroxypropanesulfonic acid (TAPSO) buffer, 2-[4-(2-hydroxyethyl)piperazin-1-yl]ethanesulfonic acid (HEPES) buffer, 2-[[1,3-dihydroxy-2-(hydroxymethyl)propan-2-yl]amino]ethanesulfonic acid (TES) buffer, 3-Morpholinopropane-1-sulfonic acid (MOPS) buffer, 1,4-Piperazinediethanesulfonic acid (PIPES) buffer, or 2-(N-morpholino)ethanesulfonic acid (MES) buffer.

30. The method of any of claims 1-25, wherein the homeopathic potency solution, polar and polarizable molecule solution, reference solution, or sample solution are prepared in the absence of light.

31. The method of any of claims 1-26, wherein the homeopathic potency solution, polar and polarizable molecule solution, reference solution, or sample solution are kept in the absence of light from preparation time until a time of spectral determination.

32. The method of any of claim 26 or 27, wherein the light is light with a wavelength of greater than 350 nm.

33. The method of any of claims 1-28, wherein the homeopathic potency solution, polar and polarizable molecule solution, reference solution, or sample solution are incubated for a period of up to 20 days prior to a spectral determination thereof.

34. The method of claim 9, wherein the ionic solvent is 1-ethyl-3-methylimidazolium acetate.

35. The method of claim 9, wherein the non-ionic solvent is water, ethanol, tert-butyl alcohol, or a combination thereof.

36. The method of any of claims 1-4 or 9-31, wherein the polar and polarizable molecule comprises a compound of the structure 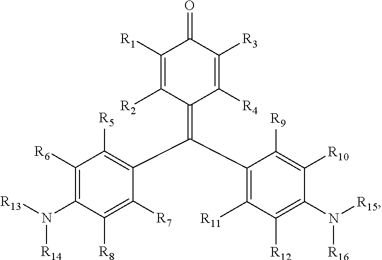
wherein R₁, R₂, R₃, R₄, R₅, R₆, R₇, R₈, R₉, R₁₀, R₁₁, R₁₂, R₁₃, R₁₄, R₁₅, and R₁₆ are each independently selected from hydrogen, alkyl, acyl, haloalky, hydroxyl, alkoxy, haloalkoxy, amino, nitro, mercapto, cyano, silyl, alkylsilyl, alkenyl, alkynyl, aryl, aralkyl, alkenoxy, alkynoxy, aryloxy, acyloxy, alkylamino, alkenylamino, alkynylamino, arylamino, amido, alkylthio, alkenylthio, alkynylthio, and arylthio.

37. The method of any of claims 1-4 or 9-31, wherein the polar and polarizable molecule comprises a compound of the structure 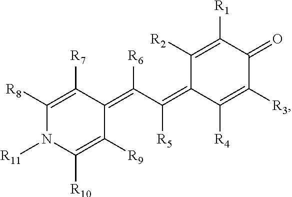
wherein R₁, R₂, R₃, R₄, R₅, R₆, R₇, R₈, R₉, R₁₀, and R₁₁ are each independently selected from hydrogen, alkyl, acyl, haloalky, hydroxyl, alkoxy, haloalkoxy, amino, nitro, mercapto, cyano, silyl, alkylsilyl, alkenyl, alkynyl, aryl, aralkyl, alkenoxy, alkynoxy, aryloxy, acyloxy, alkylamino, alkenylamino, alkynylamino, arylamino, amido, alkylthio, alkenylthio, alkynylthio, and arylthio.

38. The method of any of claims 1-33, wherein the maximum value of the difference spectrum correlates with a dipole moment of the polar and polarizable molecule.
